(19) United States
(12) Patent Application Publication
(10) Pub. No.: US 2002/0107215 A1
(43) Pub. Date: Aug. 8, 2002

(54) Tissue-associated proteins and their uses

(75) Inventors: Joseph P. Brown, Seattle, WA (US); David Pritchard, Seattle, WA (US); Vasiliki Demas, Seattle, WA (US); Glenna C. Burmer, Seattle, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: LifeSpan BioSciences, Inc., Seattle, WA

(21) Appl. No.: 09/920,302

(22) Filed: Jul. 31, 2001

Related U.S. Application Data
(60) Provisional application No. 60/222,224, filed on Aug. 1, 2000.

(51) Int. Cl. A61K 48/00; C12Q 1/68; A61K 9/127

(52) U.S. Cl. 514/44; 424/450; 435/6

(57) ABSTRACT

The present invention relates to the discovery of nucleic acids and proteins that are markers for specific normal or cancerous tissues. The identification of these markers has diagnostic uses in detecting cancer in a subject, as well as in determining the tissue of origin for a cancerous cell. These tissue-specific molecules further allow the tissue specific expression of a polynucleotide or protein of interest. In addition, the tissue-specific molecules of the invention have therapeutic applications for targeting compounds to a tissue of interest.

### CROSS-REFERENCES TO RELATED APPLICATIONS

The present application claims priority to U.S. Ser. No. 60/222,224, filed Aug. 1, 2000, herein incorporated be reference in its entirety.

### STATEMENT AS TO RIGHTS TO INVENTIONS MADE UNDER FEDERALLY SPONSORED RESEARCH AND DEVELOPMENT

Not applicable.

### BACKGROUND OF THE INVENTION

The normal functioning and development of cells and organisms require the presence of particular gene products at appropriate times and locations. While certain gene products must be present in all cells for many fundamental physiological processes to occur, other gene products are only necessary in certain cells or tissues. In addition, while some gene products are continuously present either in all cells or in certain cells or tissues, other gene products are only required at specific times during development. Moreover, certain gene products are expressed in response to a diseased state of a cell (e.g., in a cancerous cell), while expression of other gene products is inhibited in diseased cells.

The identification of genes that are expressed under specific circumstances (e.g., in diseased or healthy cells), or in different cells or tissues is thus an essential step in understanding tissue differentiation, as well as disease development. Although numerous genes associated with specific diseases or disorders, or having tissue-specificity have been identified, many more remain to be discovered.

In addition to the fundamental importance of identifying tissue-specific genes for understanding the differentiation processes of tissues, as well as for diagnostic purposes, the identification of such genes also provides essential therapeutic tools. For example, while a large number of antineoplastic drugs are available, their systemic use is associated with undesirable side effects including toxicity to normal cells, which limits the doses that can be used for treatment and, therefore, the effectiveness of the drug. As described below, the identification of tissue-specific genes allows targeting of the drug to the specific tissue that requires treatment, thus avoiding the toxicity problem and allowing the use of higher and more effective doses.

The present invention is based on the discovery that a number of polynucleotide sequences or proteins are specifically expressed in particular healthy or cancerous tissues in mammals. This discovery has provided novel and badly needed diagnostic, prognostic, and therapeutic tools for many types of cancers. In addition, this discovery has provided tools for identifying the tissue-identity of a cell, as well as tools for directing the expression of a gene of interest to a desired tissue.

### SUMMARY OF THE INVENTION

The present invention is based, at least in part, on the discovery that a variety of molecules are specifically expressed in particular healthy or cancerous tissues. Accordingly, the present invention provides methods for determining the tissue of origin for a cell, as well as methods for diagnosing cancer using the tissue-specific molecules of the invention. The present invention is further directed to fusion proteins, chimeric molecules and targeted liposomes that are useful for targeting the delivery of a molecule of interest to a desired tissue. In addition, the present invention embraces methods for identifying modulators of cancer development and for arresting cancer.

In one aspect, the present invention provides a method for expressing a molecule in a tissue of interest, the method comprising operably linking a polynucleotide encoding the molecule of interest to a tissue-specific promoter, wherein the tissue-specific promoter is the promoter of a gene selected from the genes set forth in Table 1A and B. The molecule thus expressed can be a protein or a nucleic acid.

In a second aspect, the present invention provides a method for diagnosing cancer in a subject, the method comprising detecting the expression of a cancer-associated molecule according to Table 1B in a tissue of interest in the subject, wherein expression of the molecule is indicative of cancer of the tissue of interest. In one embodiment, the cancer-associated molecule is an mRNA. In another embodiment, the cancer-associated molecule is detected in an immunoassay.

The present invention also provides a method for determining the tissue of origin for a cell, the method comprising detecting the expression in the cell of a tissue-specific molecule selected from the group consisting of the molecules set forth in Table 1A and B. In one embodiment, the detected tissue-specific molecule is an mRNA. In another embodiment, the tissue-specific molecule is detected in an immunoassay. In some embodiments, the cell is a cancerous cell.

The present invention is further directed to fusion proteins comprising at least a portion of a protein, wherein the protein specifically binds to a target protein selected from the group consisting of the proteins encoded by the polynucleotides set forth in Table 1A and B, wherein the portion is sufficient to confer specific binding to the target protein, and a second, heterologous protein. In a preferred embodiment, the second, heterologous protein is a therapeutic protein. In one embodiment, the therapeutic protein is a toxin.

In addition, the present invention is directed to targeted chimeric molecules comprising at least a portion of a molecule, wherein the molecule specifically binds to a target molecule selected from the group consisting of the ribonucleotides and the proteins encoded by the polynucleotides set forth in Table 1A and B, wherein the portion is sufficient to confer specific binding to the target molecule, and a therapeutic agent. In a preferred embodiment, the therapeutic drug is a cytotoxic agent. The targeted chimeric molecules of the invention can be used for delivering a therapeutic agent to a tissue of interest in a patient in need thereof. Moreover, the targeted chimeric molecule of the invention can also be used for eliminating a tissue in a patient in need thereof. Accordingly, methods for delivering a therapeutic agent to a tissue of interest or methods for eliminating a tissue of interest using the described targeted chimeric molecules are also embraced by the present invention. In some embodiments, the tissue of interest is a cancerous tissue and the targeted chimeric molecules comprise at least a portion of a molecule, wherein said molecule specifically binds to a target molecule selected from the group consisting of the ribonucleotides and the proteins encoded by the polynucleotides set forth in Table 1B, wherein the portion is sufficient to confer specific binding to the target molecule.

In yet another aspect, the present invention provides targeted therapeutic liposomes comprising at least a portion of a molecule, wherein the molecule specifically binds to a target molecule selected from the group consisting of the ribonucleotides and the proteins encoded by the polynucleotides set forth in Table 1A and B, wherein the portion is sufficient to confer specific binding to the target molecule, and a therapeutic liposome. In a preferred embodiment, the therapeutic liposome comprises a cytotoxic agent. The present invention also encompasses methods for delivering a therapeutic agent to a specific tissue in a patient in need thereof using the described targeted therapeutic liposomes. The present invention further provides a method for eliminating a tissue in a patient in need thereof, the method comprising administering to the patient a targeted therapeutic liposome as described herein, wherein the targeted liposome comprises a cytotoxic agent. In some embodiments, the tissue is a cancerous tissue and the therapeutic liposomes are targeted using at least a portion of a molecule, wherein the molecule specifically binds to a target molecule selected from the group consisting of the ribonucleotides and the proteins encoded by the polynucleotides set forth in Table 1B, wherein the portion retains specific binding to the target molecule.

The present invention also embraces a method for arresting cancer, the method comprising inhibiting a cancer-associated molecule according to Table 1B. In one embodiment, the cancer-associated molecule is inhibited using an antisense polynucleotide. In another embodiment, the cancer-associated molecule is inhibited using an antibody that specifically binds to the cancer-associated molecule.

In additional aspects, the present invention provides methods for identifying a modulator of cancer development in a cell, the method comprising culturing the cell in the presence of the modulator to form a first cell culture, contacting RNA or cDNA from the first cell culture with a probe which comprises a polynucleotide sequence selected from the sequences set forth in Table 1B, determining whether the amount of the probe which hybridizes to the RNA or cDNA from the first cell culture is increased or decreased relative to the amount of the probe which hybridizes to RNA or cDNA from a second cell culture grown in the absence of said modulator, and detecting the presence or absence of a decreased proliferative potential, transformation or malignancy in the first cell culture relative to the second cell culture. Such modulators are useful for modulating cancer development. Accordingly, the present invention provides a method for modulating cancer development in a patient in need thereof, the method comprising administering to the patient a modulator of cancer development as described above, wherein the modulator decreases the expression of a polynucleotide sequence selected from the group of sequences set forth in Table 1B.

In still another aspect, the present invention provides kits for carrying out the various methods. For instance, in one embodiment, a kit is provided for determining the tissue of origin for a cell, the kit comprising at least one probe, each probe comprising at least about 10 nucleotides from a polynucleotide sequence selected from the group consisting of the sequences listed in Table 1A and B, and a label or labels for detecting the presence of the probe(s). Similarly, a kit is provided for detecting whether a cell is undergoing transformation or becoming malignant, the kit comprising: a probe which comprises a polynucleotide sequence associated with cancer, comprising at least about 10 nucleotides from a polynucleotide sequence selected from the group consisting of the sequences listed in Table 1B; and a label for detecting the presence of the probe. Additionally, this kit can further comprise a plurality of probes each of which comprises a polynucleotide sequence associated with cancer; and a label or labels for detecting the presence of the plurality of probes. The probes of the kits of the invention can optionally be immobilized on a solid support (e.g., a chip).

The invention also embraces the use of antisense methods for examining cancer in animals and cells. Typically, any time a gene is identified, it can be examined by knocking out the gene in an animal and observing the effect on the animal phenotype. Knockouts can be achieved by transposons which insert by homologous recombination, antisense or ribozymes specifically directed at disturbing the embryonic stem cells of an organism such as a mouse. Ribozymes can include any of the various types of ribozymes modified to cleave the mRNA encoding, for example, a cancer-associated protein of interest. Examples include hairpins and hammerhead ribozymes. Finally, antisense molecules which selectively bind, for example, to the mRNA encoding a cancer-associated protein of interest are expressed via expression cassettes operably linked to subsequences of the cancer-associated gene and generally comprise 20-50 base long sequences in opposite orientation to the mRNA to which they are targeted.

### BRIEF DESCRIPTION OF THE DRAWINGS

Not applicable.

### DESCRIPTION OF THE SPECIFIC EMBODIMENTS

I. Introduction

The present invention is based, at least in part, on the identification of polynucleotides and polypeptides that show tissue-specificity. The polynucleotides and polypeptides of the invention can thus serve as “markers” for the tissue(s) where they are specifically expressed. Some polynucleotides and polypeptides of the invention are expressed specifically in one or more particular tissue(s) in healthy individuals. Other polynucleotides and polypeptides of the invention are specifically expressed in cancerous tissues.

The present invention provides methods for determining the identity of a cell (or group of cells) using the markers described herein. In particular, the markers can be used for diagnosing whether a cell is in a cancerous state. Furthermore, the markers of the invention are useful for determining the tissue of origin for a cell, in particular for a cancerous cell (e.g., a metastasis).

The present invention also provides methods for targeting therapeutic compounds to a tissue of interest. For example, a tissue-specific promoter from a marker of the invention can be used to specifically drive the expression of a compound in a particular tissue. Alternatively, fusion proteins and targeted liposomes can be constructed using molecules that specifically bind to the marker molecules of the present invention and such fusion proteins and targeted liposomes can then be used for targeting therapeutic compounds to a tissue of interest.

II. Definitions

The term “tissue-specificity” refers to the property of a polynucleotide or polypeptide to be expressed exclusively, or at a significantly higher level, in one or several particular tissue(s). A “tissue-specific molecule” refers to a polynucleotide or a protein having “tissue-specificity.” The presence of such “tissue-specific” polynucleotides or proteins can thus serve as a “marker” for the tissue(s) where they are expressed. In the context of the present invention, the “tissue-specific molecules” are, therefore, also referred to as “markers.”

“Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize), as well as any of a number of characteristic structural and/or molecular features. A “cancerous” or “malignant cell” is understood as a cell having specific structural properties, lacking differentiation and being capable of invasion and metastasis. Examples of cancers are breast, colon, kidney, liver, ovarian and prostate cancer.

“Neoplasia” or “tumor” describe new abnormal growth which can be benign or malignant. “Neoplastic proliferation” describes cell proliferation that persists even in the absence of growth stimulus.

In the context of the invention, the term “transformation” refers to the change that a normal cell undergoes as it becomes malignant. In eukaryotes, the term “transformation” can be used to describe the conversion of normal cells to malignant cells in cell culture.

“Proliferating cells” are those which are actively undergoing cell division and growing exponentially. “Loss of cell proliferation control” refers to the property of cells that have lost the cell cycle controls that normally ensure appropriate restriction of cell division. Cells that have lost such controls proliferate at a faster than normal rate, without stimulatory signals, and do not respond to inhibitory signals.

“Amplification primers” are oligonucleotides comprising either natural or analog nucleotides that can serve as the basis for the amplification of a selected nucleic acid sequence. They include, for example, both polymerase chain reaction primers and ligase chain reaction oligonucleotides.

The term “antibody” refers to a polypeptide substantially encoded by an immunoglobulin gene or immunoglobulin genes, or fragments thereof which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see, Paul (Ed.) Fundamental Immunology, Third Edition, Raven Press, NY (1993)). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies (e.g., single chain Fv).

“Biological samples” refers to any tissue or liquid sample having genomic DNA or other nucleic acids (e.g., mRNA) or proteins. It refers to samples of cells or tissue from a normal healthy individual as well as samples of cells or tissue from a subject suspected of having cancer, e.g., breast cancer, colon cancer, kidney cancer, liver cancer, ovarian cancer, prostate cancer, etc.

The term “isolated,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein which is the predominant species present in a preparation is substantially purified. In particular, an isolated gene is separated from open reading frames which flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “gene” means the segment of DNA involved in producing a polypeptide chain; it includes regions preceding and following the coding region (leader and trailer) as well as intervening sequences (introns) between individual coding segments (exons).

“Nucleic acid” refers to deoxyribonucleotides or ribonucleotides and polymers thereof in either single- or double-stranded form. Unless specifically limited, the term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). The term also encompasses ribonucleotides including HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Unless otherwise indicated, a particular nucleic acid sequence also implicitly encompasses conservatively modified variants thereof (e.g., degenerate codon substitutions) and complementary sequences, as well as the sequence explicitly indicated. Polynucleotide variants may contain one or more substitutions, additions, deletions and/or insertions, as further described below, preferably such that the tissue-specificity of the encoded polypeptide is not affected relative to the native polypeptide. The effect on the expression of the encoded polypeptide may generally be assessed as described herein. The term “variants” also encompasses interspecies homologs. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081(1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The phrase “a nucleic acid sequence encoding” refers to a nucleic acid which contains sequence information for a structural RNA such as rRNA, a tRNA, or for the primary amino acid sequence of a specific protein or peptide, or for a binding site for a trans-acting regulatory agent. This phrase specifically encompasses degenerate codons (i.e., different codons which encode a single amino acid) of the native sequence or sequences which may be introduced to conform with codon preference in a specific host cell.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full length proteins, wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. “Amino acid analogs” refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

A polypeptide “variant,” as used herein, is a polypeptide that differs from the recited polypeptide only in conservative substitutions and/or modifications, such that the tissue-specificity properties of the polypeptide are retained. Polypeptide variants preferably exhibit at least about 70%, more preferably at least about 90%, and most preferably at least about 95% identity or more to the identified polypeptides.

A “conservative substitution” applies to both amino acid and nucleic acid sequences. With respect to particular nucleic acid sequences, the term “conservative substitutions” refers to changes in the nucleic acid sequence that result in nucleic acids encoding identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservative substitution” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

(see, e.g., Creighton, Proteins (1984)).

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic acid concentration) at which 50% of the probes complementary to the target hybridize to the target sequence at equilibrium (as the target sequences are present in excess, at Tₘ 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3, and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For stringent hybridization, a positive signal is at least twice background, preferably 10 times background hybridization. Exemplary stringent hybridization conditions include: 50% formamide, 5× SSC and 1% SDS incubated at 42° C. or 5× SSC and 1% SDS incubated at 65° C., with a wash in 0.2× SSC and 0.1% SDS at 65° C.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such case, the nucleic acids typically hybridize under moderately stringent hybridization conditions. Exemplary “moderately” stringent hybridization conditions include a hybridization in a buffer of 40% formamide, 1 M NaCl, 1% SDS at 37° C., and a wash in 1× SSC at 45° C. A positive hybridization is at least twice background. Those of ordinary skill will readily recognize that alternative hybridization and wash conditions can be utilized to provide conditions of similar stringency.

Another indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Yet another indication that two nucleic acid sequences are substantially identical is that the same primers can be used to amplify the sequence.

The terms “identical” or percent “identity,” in the context of two or more nucleic acids or polypeptide sequences, refer to two or more sequences or subsequences that are the same or have a specified percentage of amino acid residues or nucleotides that are the same (i.e., 55%, 60%, 65%, 70%, 75%, or 80% identity, preferably 85%, 90%, 95%, 96%, 97%, 98%, 99% or higher identity over a specified window region), when compared and aligned for maximum correspondence over a comparison window or designated region, as measured using one of the following sequence comparison algorithms or by manual alignment and visual inspection. Such sequences are then said to be “substantially identical.” This definition also refers to the complement of a test sequence. Preferably, the identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, A model of evolutionary change in proteins—Matrices for detecting distant relationships, In: Dayhoff (ed.) Atlas of Protein Sequence and Structure, National Biomedical Research Foundation, Washington D.C. Vol. 5, Suppl. 3, pp. 345-358 (1978); Hein, Unified Approach to Alignment and Phylogenes pp. 626-645 Methods in Enzymology vol. 183, Academic Press, Inc., San Diego, Calif. (1990); Higgins and Sharp, CABIOS 5:151-153 (1989); Myers and Muller, CABIOS 4:11-17 (1988); Robinson, Comb. Theor 11:105 (1971); Santou and Nes, Mol. Biol. Evol. 4:406-425 (1987); Sneath and Sokal, Numerical Taxonomy—the Principles and Practice of Numerical Taxonomy, Freeman Press, San Francisco, Calif. (1973); Wilbur and Lipman, Proc. Natl. Acad. Sci. USA 80:726-730 (1983).

Alternatively, optimal alignment of sequences for comparison can be conducted, e.g., by the local homology algorithm of Smith and Waterman, Adv. Appl. Math. 2:482 (1981), by the homology alignment algorithm of Needleman and Wunsch, J. Mol. Biol. 48:443 (1970), by the search for similarity method of Pearson and Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. (1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=-4 and a comparison of both strands. For amino acid sequences, the BLASTP program uses as defaults a wordlength of 3, and expectation (E) of 10, and the BLOSUM62 scoring matrix (see, Henikoff and Henikoff, Proc. Natl. Acad. Sci. USA 89:10915 (1989)) alignments (B) of 50, expectation (E) of 10, M=5, N=-4, and a comparison of both strands.

The BLAST algorithm also performs a statistical analysis of the similarity between two sequences (see, e.g., Karlin and Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

As used herein a “nucleic acid probe” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions.

Nucleic acid probes can be DNA or RNA fragments. DNA fragments can be prepared, for example, by digesting plasmid DNA, or by use of PCR, or synthesized by either the phosphoramidite method described by Beaucage and Carruthers (Tetrahedron Lett. 22:1859-1862 (1981)), or by the triester method according to Matteucci et al. (J. Am. Chem. Soc. 103:3185 (1981)). A double-stranded fragment may then be obtained, if desired, by annealing the chemically synthesized single strands together under appropriate conditions, or by synthesizing the complementary strand using DNA polymerase with an appropriate primer sequence. Where a specific sequence for a nucleic acid probe is given, it is understood that the complementary strand is also identified and included. The complementary strand will work equally well in situations where the target is a double-stranded nucleic acid.

A “labeled nucleic acid probe” is a nucleic acid probe that is bound, either covalently, through a linker, or through ionic, van der Waals or hydrogen bonds to a label such that the presence of the probe may be determined by detecting the presence of the label bound to the probe.

The term “recombinant” when used with reference, e.g., to a cell, or to a nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under-expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The terms “fusion polypeptide” or “fusion protein” refer to a protein having at least two heterologous polypeptides covalently linked, either directly or via an amino acid linker. Typically, a linker has no specific biological activity other than to join the different polypeptides or to preserve some minimum distance or other spatial relationship between them. However, the constituent amino acids of a linker may be selected to influence some property of the molecule such as the folding, net charge, or hydrophobicity. The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order. This term also refers to conservatively modified variants, polymorphic variants, alleles, mutants, subsequences, and interspecies homologs of the polypeptides that make up the fusion protein.

The phrase “specifically (or selectively) binds to an antibody” or “specifically (or selectively) immunoreactive with,” when referring to a protein or peptide, refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein. For example, antibodies raised against a protein of the invention can be selected to obtain antibodies specifically immunoreactive with that protein and not with other proteins, except for polymorphic variants. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein. See, Harlow and Lane Antibodies, A Laboratory Manual, Cold Spring Harbor Publications, NY (1988) for a description of immunoassay formats and conditions that can be used to determine specific immunoreactivity. Typically, a specific or selective reaction will be at least twice the background signal or noise and more typically more than 10 to 100 times background.

The phrase “portion sufficient to confer specific binding” refers to any portion of a molecule (e.g., polynucleotide or protein) that retains the same “binding specificity” as the entire molecule, i.e., that specifically binds to the same target molecule. Portions of a polynucleotide or of a protein can be easily obtained using standard techniques well-known to those of skill in the art. The binding-specificity of the resulting portions can then be tested using the methods described infra.

In the context of the present invention, “chimeric molecule” refers to a molecule comprising two or more different types of molecules that exist separately in their native state and that are joined together to form a single molecule having the desired functionality of all of its constituent molecules. The individual components of a single “chimeric molecule” can be molecules of different natures such as, e.g., polypeptides, nucleic acids, radioactive isotopes, pharmaceutical agents, hormones, etc.

“Targeted therapeutic compounds” are “chimeric molecules” typically comprising a therapeutic compound coupled to a “targeting moiety” that targets the compound to a tissue of interest. In the context of the present invention, the term “targeting moiety” refers to a molecule that specifically binds to a tissue-specific molecule of the invention, or to a portion thereof that retains specific binding to the target molecule of the invention. Within a “targeted therapeutic compound,” the targeting moiety and the therapeutic compound portions can, in some cases, be attached via a “linker.” As used herein, the term “linker” refers to a molecule that joins the different components of the “targeted therapeutic compound.”

In the context of the present invention, the term “therapeutic liposome” refers to a liposome which includes a therapeutic agent entrapped in the liposome.

III. General Recombinant Nucleic Acids Methods for Use with the Invention

In numerous embodiments of the present invention, nucleic acids encoding the tissue-specific proteins (i.e., the marker proteins) of interest will be isolated and cloned using recombinant methods. Such embodiments are used, e.g., to isolate marker protein-encoding polynucleotides for protein expression or during the generation of variants, derivatives, expression cassettes, or other sequences derived from the marker proteins, to monitor tissue-specific or cancer-associated gene expression, for the isolation or detection of tissue-specific sequences in different species, for diagnostic purposes in a patient, e.g., to detect mutations in cancer-associated genes, etc. In one embodiment, the nucleic acids of the invention are from any mammal, including, in particular, e.g., a human, a primate, a rat, a mouse, etc.

A. General Recombinant Nucleic Acids Methods

The numerous applications of the present invention involving the cloning, synthesis, maintenance, mutagenesis, and other manipulations of nucleic acid sequences can be performed using routine techniques in the field of recombinant genetics. Basic texts disclosing the general methods of use in this invention include Sambrook et al., Molecular Cloning: A Laboratory Manual (2nd ed. 1989); Kriegler, Gene Transfer and Expression: A Laboratory Manual (1990); and Ausubel et al., Current Protocols in Molecular Biology (1994). Unless otherwise stated all enzymes are used in accordance with the manufacturer's instructions.

Nucleotide sizes are given in either kilobases (kb) or base pairs (bp). These are estimates derived from agarose or acrylamide gel electrophoresis or, alternatively, from published DNA sequences.

Oligonucleotides that are not commercially available can be chemically synthesized according to the solid phase phosphoramidite triester method first described by Beaucage and Caruthers, Tetrahedron Letts. 22(20):1859-1862 (1981), using an automated synthesizer, as described in Needham Van Devanter et al., Nucleic Acids Res. 12:6159-6168 (1984). Purification of oligonucleotides is, for example, by either native acrylamide gel electrophoresis or by anion-exchange HPLC as described in Pearson and Reanier, J. Chrom. 255:137-149 (1983).

The nucleic acids described herein, or fragments thereof, can be used as hybridization probes for genomic or cDNA libraries to isolate the corresponding complete gene (including regulatory and promoter regions, exons and introns) or cDNAs, in particular cDNA clones corresponding to full-length transcripts. The probes may also be used to isolate other genes and cDNAs which have a high sequence identity to the gene of interest or similar biological activity. Probes of this type preferably have at least 30 bases and may contain, for example, 50 or more bases.

The sequence of the cloned genes and synthetic oligonucleotides can be verified using the chemical degradation method of Maxam and Gilbert, Methods in Enzymology 65:499-560 (1980). The sequence can be confirmed after the assembly of the oligonucleotide fragments into the double-stranded DNA sequence using the method of Maxam and Gilbert, supra, or the chain termination method for sequencing double-stranded templates of Wallace et al., Gene 16:21-26 (198 1). Southern blot hybridization techniques can be carried out according to Southern et al., J. Mol. Biol. 98:503 (1975).

B. Cloning Methods for the Isolation of Nucleotide Sequences Encoding the Desired Proteins

In general, the nucleic acids encoding the subject proteins are cloned from DNA sequence libraries that are made to encode copy DNA (cDNA) or genomic DNA. The particular sequences can be located by hybridizing with an oligonucleotide probe, the sequence of which can be derived from the sequences provided herein (e.g., the sequences set forth in Table 1A and B), which provides a reference for PCR primers and defines suitable regions for isolating tissue-specific probes (including, e.g., cancer-associated probes). Alternatively, where the sequence is cloned into an expression library, the expressed recombinant protein can be detected immunologically with antisera or purified antibodies made against the tissue-specific protein of interest.

Methods for making and screening genomic and cDNA libraries are well-known to those of skill in the art (see, e.g., Gubler and Hoffman, Gene 25:263-269 (1983); Benton and Davis, Science 196:180-182 (1977); and Sambrook et al., supra).

Briefly, to make the cDNA library, one should choose a source that is rich in mRNA. The mRNA can then be made into cDNA, ligated into a recombinant vector, and transfected into a recombinant host for propagation, screening and cloning. For a genomic library, the DNA is extracted from a suitable tissue and either mechanically sheared or enzymatically digested to yield fragments of preferably about 5-100 kb. The fragments are then separated by gradient centrifugation from undesired sizes and are constructed in bacteriophage lambda vectors. These vectors and phage are packaged in vitro, and the recombinant phages are analyzed by plaque hybridization. Colony hybridization is carried out as generally described in Grunstein et al., Proc. Natl. Acad. Sci. USA 72:3961-3965 (1975).

An alternative method combines the use of synthetic oligonucleotide primers with polymerase extension on an mRNA or DNA template. Suitable primers can be designed from specific marker molecules (e.g., marker molecules having a sequence selected from the sequences described in Table 1A and B). This polymerase chain reaction (PCR) method amplifies the nucleic acids encoding a protein of interest directly from mRNA, cDNA, genomic libraries or cDNA libraries. Restriction endonuclease sites can be incorporated into the primers. Polymerase chain reaction or other in vitro amplification methods may also be useful, for example, to clone nucleic acids encoding specific proteins and express said proteins, to synthesize nucleic acids that will be used as probes for detecting the presence of mRNA encoding a marker protein of the invention in physiological samples, for nucleic acid sequencing, or for other purposes (see, e.g., U.S. Pat. Nos. 4,683,195 and 4,683,202). Genes amplified by a PCR reaction can be purified, e.g., from agarose gels, and cloned into an appropriate vector.

Appropriate primers and probes for identifying the genes encoding the tissue-specific proteins of the invention from mammalian tissues can be derived from the sequences provided herein, in particular the sequences set forth in Table 1A and B. For a general overview of PCR, see, Innis et al., PCR Protocols: A Guide to Methods and Applications, Academic Press, San Diego (1990).

Synthetic oligonucleotides can be used to construct genes. This is done using a series of overlapping oligonucleotides, usually 40-120 bp in length, representing both the sense and anti-sense strands of the gene. These DNA fragments are then annealed, ligated and cloned.

A gene encoding a tissue-specific protein of the invention can be cloned using intermediate vectors before transformation into mammalian cells for expression. These intermediate vectors are typically prokaryotic vectors or shuttle vectors. The proteins can be expressed in either prokaryotes, using standard methods well-known to those of skill in the art, or eukaryotes as described infra.

C. Expression in Eukaryotes

Standard eukaryotic transfection methods are used to produce eukaryotic cell lines, e.g., yeast, insect, or mammalian cell lines, which express large quantities of the tissue-specific proteins of the invention which are then purified using standard techniques (see, e.g., Colley et al., J. Biol. Chem. 264:17619-17622 (1989); and Guide to Protein Purification, in Vol. 182 of Methods in Enzymology, Deutscher ed., (1990)).

Transformations of eukaryotic cells are performed according to standard techniques as described by Morrison, J. Bact. 132:349-351 (1977), or by Clark-Curtiss and Curtiss, Methods in Enzymology 101:347-362, R. Wu et al. (Eds) Academic Press, NY (1983).

Any of the well-known procedures for introducing foreign nucleotide sequences into host cells may be used. These include the use of calcium phosphate transfection, polybrene, protoplast fusion, electroporation, liposomes, microinjection, plasma vectors, viral vectors and any of the other well-known methods for introducing cloned genomic DNA, cDNA, synthetic DNA or other foreign genetic material into a host cell (see Sambrook et al., supra). It is only necessary that the particular genetic engineering procedure utilized be capable of successfully introducing at least one gene into the host cell which is capable of expressing the protein.

The particular eukaryotic expression vector used to transport the genetic information into the cell is not particularly critical. Any of the conventional vectors used for expression in eukaryotic cells may be used. Expression vectors containing regulatory elements from eukaryotic viruses are typically used. Suitable vectors for use in the present invention include, but are not limited to, SV40 vectors, vectors derived from bovine papilloma virus or from the Epstein Barr virus and baculovirus vectors, and any other vector allowing expression of proteins under the direction of the SV-40 later promoter, metallothionein promoter, murine mammary tumor virus promoter, Rous sarcoma virus promoter, polyhedrin promoter, or other promoters shown effective for expression in eukaryotic cells.

The vectors usually include selectable markers which result in gene amplification, such as, e.g., thymidine kinase, aminoglycoside phosphotransferase, hygromycin B phosphotransferase, xanthine-guanine phosphoribosyl transferase, CAD (carbamyl phosphate synthetase, aspartate transcarbamylase, and dihydroorotase), adenosine deaminase, dihydrofolate reductase, asparagine synthetase and ouabain selection. Alternatively, high yield expression systems not involving gene amplification are also suitable, such as, e.g., using a baculovirus vector in insect cells, with a target protein encoding sequence under the direction of the polyhedrin promoter or other strong baculovirus promoters.

The expression vector of the present invention will typically contain both prokaryotic sequences that facilitate the cloning of the vector in bacteria as well as one or more eukaryotic transcription units that are expressed only in eukaryotic cells, such as mammalian cells. The vector may or may not comprise a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the transfected DNA integrates into the genome of the transfected cell, where the promoter directs expression of the desired gene. The expression vector is typically constructed from elements derived from different, well characterized viral or mammalian genes. For a general discussion of the expression of cloned genes in cultured mammalian cells, see, Sambrook et al., supra, Ch. 16.

The prokaryotic elements that are typically included in the mammalian expression vector include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells.

The expression vector contains a eukaryotic transcription unit or expression cassette that contains all the elements required for the expression of the DNA encoding the tissue-specific protein of interest in eukaryotic cells. A typical expression cassette contains a promoter operably linked to the DNA sequence encoding the tissue-specific protein of interest and signals required for efficient polyadenylation of the transcript. The DNA sequence encoding the protein of interest may typically be linked to a cleavable signal peptide sequence to promote secretion of the encoded protein by the transformed cell. Such signal peptides would include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues (see, Enhancers and Eukaryotic Expression, Cold Spring Harbor Press, Cold Spring Harbor, N.Y. (1983)).

In the construction of the expression cassette, the promoter is preferably positioned at about the same distance from the heterologous transcription start site as it is from the transcription start site in its natural setting. As is known in the art, some variation in this distance can, however, be accommodated without loss of promoter function.

In addition to a promoter sequence, the expression cassette should also contain a transcription termination region downstream of the structural gene to provide for efficient termination. The termination region may be obtained from the same gene as the promoter sequence or may be obtained from a different gene.

If the mRNA encoded by the structural gene is to be efficiently translated, polyadenylation sequences are also commonly added to the vector construct. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40, or a partial genomic copy of a gene already resident on the expression vector.

In addition to the elements already described, the expression vector of the present invention may typically contain other specialized elements intended to increase the level of expression of cloned genes or to facilitate the identification of cells that carry the transfected DNA. For instance, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The cDNA encoding the protein of interest can be ligated to various expression vectors for use in transforming host cell cultures. The vectors typically contain gene sequences to initiate transcription and translation of the tissue-specific gene of interest. These sequences need to be compatible with the selected host cell. In addition, the vectors preferably contain a marker to provide a phenotypic trait for selection of transformed host cells such as dihydrofolate reductase or metallothionein. Additionally, a vector might contain a replicative origin.

Cells of mammalian origin are illustrative of cell cultures useful for the production of, for example, a tissue-specific protein of interest. Mammalian cell systems often will be in the form of monolayers of cells, although mammalian cell suspensions may also be used. Illustrative examples of mammalian cell lines include VERO and HeLa cells, NIH 3T3, COS, Chinese hamster ovary (CHO), W138, BHK, COS-7 or MDCK cell lines.

As indicated above, the vector, e.g., a plasmid, which is used to transform the host cell, preferably contains DNA sequences to initiate transcription and sequences to control the translation of the gene sequence encoding the marker protein of interest. These sequences are referred to as expression control sequences. Illustrative expression control sequences are described, e.g., in Berman et al., Science 222:524-527 (1983); Thomsen et al., Proc. Natl. Acad. Sci. 81:659-663 (1984); and Brinster et al., Nature 296:39-42 (1982). The cloning vector containing the expression control sequences is cleaved using restriction enzymes, adjusted in size as necessary or desirable and ligated with sequences encoding the marker protein by means well-known in the art.

When higher animal host cells are employed, polyadenylation or transcription terminator sequences from known mammalian genes need to be incorporated into the vector. An example of a terminator sequence is the polyadenylation sequence from the bovine growth hormone gene. Sequences for accurate splicing of the transcript may also be included. An example of a splicing sequence is the VP 1 intron from SV40 (Sprague et al., J. Virol. 45:773-781 (1983)).

Additionally, gene sequences to control replication in the host cell may be incorporated into the vector such as those found in bovine papilloma virus type-vectors (see, Saveria-Campo, “Bovine Papilloma virus DNA a Eukaryotic Cloning Vector” In: DNA Cloning Vol.II. a Practical Approach (Glover Ed.), IRL Press, Arlington, Va. pp. 213-238 (1985)).

The transformed cells are cultured by means well-known in the art. For example, such means are published in Biochemical Methods in Cell Culture and Virology, Kuchler, Dowden, Hutchinson and Ross, Inc. (1977). The expressed protein is isolated from cells grown as suspensions or as monolayers. The latter are recovered by well-known mechanical, chemical or enzymatic means.

D. Tissue-Specific Targeting of Protein Expression

The present invention also provides tissue-specific promoters useful for driving the expression of heterologous proteins in specific tissues. In particular, the tissue-specific promoters of the invention can be used for targeting therapeutic compounds to a tissue of interest.

1. Cloning a Promoter Sequence

The tissue-specific promoter sequences of the invention are preferably those from the genes comprising the polynucleotides set forth in Table 1A and B. Preferably, the promoter sequences are specific for one tissue (e.g., a healthy or a cancerous tissue).

In general, the nucleic acid sequences corresponding to a promoter of interest and related nucleic acid sequence homologs are cloned from genomic DNA libraries by hybridization with a probe, or isolated using amplification techniques with oligonucleotide primers, following the methods for cloning nucleic acids described supra.

For instance, tissue-specific promoter sequences can be isolated from genomic libraries by hybridizing the library with a nucleic acid probe corresponding to the sequence of a polynucleotide set forth in Table 1A and B. Promoter sequence polymorphic variants, alleles, and interspecies homologs that are substantially identical to the promoter sequence of interest can be isolated using promoter sequence nucleic acid probes, and oligonucleotides under stringent hybridization conditions, by screening genomic libraries, as described supra.

Amplification techniques using primers can also be used to amplify and isolate a polynucleotide sequence corresponding to a tissue-specific promoter of interest from DNA (see, e.g., U.S. Pat. Nos. 4,683,195 and 4,683,202; PCR Protocols: A Guide to Methods and Applications, Innis et al., eds. (1990)). Degenerate primers derived, for example, from the sequences of the polynucleotides set forth in Table 1A and B can be used to amplify a promoter sequence (see, e.g., Dieffenfach and Dveksler, PCR Primer. A Laboratory Manual (1995)). These primers can be used, e.g., to amplify either the entire promoter sequence or a probe which is then used to screen a genomic library for the entire promoter sequence. Methods such as polymerase chain reaction (PCR) and ligase chain reaction (LCR) can be used to amplify promoter nucleic acid sequences directly from genomic libraries, following methods described supra. Restriction endonuclease sites can be incorporated into the primers to facilitate cloning.

2. Expression of Heterologous Sequences

A protein of interest can be targeted for expression in a particular tissue (e.g., a healthy or a cancerous tissue) by being expressed under the control of a promoter specific for the tissue of interest. In preferred embodiments, the protein of interest has therapeutic properties. In one embodiment, the protein of interest is a toxin.

The tissue-specific expression of natural or synthetic nucleic acids is typically achieved by constructing an expression cassette containing nucleic acid sequences encoding the protein of interest operably linked to a suitable tissue-specific promoter into an expression vector. Expression of a heterologous protein of interest under the control of a tissue-specific promoter of the invention can be performed according to standard recombinant protein expression methods, as described supra.

IV. Purification of the Proteins for Use with the Invention

After expression, the proteins of the present invention can be purified to substantial purity by standard techniques, including selective precipitation with substances as ammonium sulfate, column chromatography, immunopurification methods, and other methods known to those of skill in the art (see, e.g., Scopes, Protein Purification: Principles and Practice, Springer-Verlag, NY (1982); U.S. Pat. No. 4,673,641; Ausubel et al., supra; and Sambrook et al., supra).

A number of conventional procedures can be employed when a recombinant protein is being purified. For example, proteins having established molecular adhesion properties can be reversibly fused to the subject protein. With the appropriate ligand, a marker protein of interest, for example, can be selectively adsorbed to a purification column and then freed from the column in a relatively pure form. The fused protein is then removed by enzymatic activity. Finally, the marker proteins of the invention can be purified using immunoaffinity columns.

A. Purification of Proteins from Recombinant Bacteria

When recombinant proteins are expressed by the transformed bacteria in large amounts, typically after promoter induction, although expression can be constitutive, the proteins may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells typically, but not limited to, by incubation in a buffer of about 100-150 μg/ml lysozyme and 0.1% Nonidet P40, a non-ionic detergent. The cell suspension can be ground using a Polytron grinder (Brinkman Instruments, Westbury, N.Y.). Alternatively, the cells can be sonicated on ice. Alternate methods of lysing bacteria are described in Ausubel et al., and Sambrook et al., both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents which are capable of solubilizing aggregate-forming proteins, such as SDS (sodium dodecyl sulfate) and 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques.

Alternatively, it is possible to purify proteins from bacteria periplasm. Where the protein is exported into the periplasm of the bacteria, the periplasmic fraction of the bacteria can be isolated by cold osmotic shock in addition to other methods known to those of skill in the art (see, Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well-known to those of skill in the art.

B. Standard Protein Separation Techniques For Purifying Proteins

1. Solubility Fractionation

Often as an initial step, and if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well-known to those of skill in the art and can be used to fractionate complex protein mixtures.

2. Size Differential Filtration

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

3. Column Chromatography

The proteins of interest can also be separated from other proteins on the basis of their size, net surface charge, hydrophobicity and affinity for ligands. In addition, antibodies raised against proteins can be conjugated to column matrices and the proteins immunopurified. All of these methods are well-known in the art.

It will be apparent to one of skill that chromatographic techniques can be performed at any scale and using equipment from many different manufacturers (e.g., Pharmacia Biotech).

V. Detection of Gene Expression and Genomic Analysis of the Tissue-Specific Proteins of the Invention

The polynucleotides and polypeptides of the present invention can be employed as research reagents and materials for discovery of treatments and diagnostics to human disease. In particular, the polynucleotides and polypeptides of the present invention can be used as markers for specific tissues, both healthy and cancerous. The present invention also provides methods for detecting nucleic acids associated with cancer. It will be readily apparent to those of skill in the art that similar methods can be used to detect nucleic acids associated with, e.g., cell proliferation, cell transformation, neoplasia, metastasis, etc.

As should be apparent to those of skill in the art, the present invention is based, at least in part, in the identification of tissue-specific polynucleotides and polypeptides. In addition, the present invention is also based, in part, in the identification of cancer-associated molecules. Accordingly, the present invention provides methods for detecting the presence of such tissue-specific markers, e.g., in physiological samples or in tissue culture cells, in order to determine the identity of a tissue or cell. These methods can be used to determine the tissue of origin of a biological specimen such as, for example, a biopsy from a cancerous tissue of unknown origin, or a metastasis. Furthermore, methods are also provided for detecting the presence, alteration or absence of the cancer-associated nucleic acids of the invention (e.g., DNA or RNA) in a physiological specimen in order to determine the level of proliferation and/or transformation of cells in vitro, or ex vivo, as well as the genotype and risk of cancer development associated with mutations created in cancer-associated sequences. Although any tissue having cells bearing the genome of an individual or RNA can be used for diagnosing cancer, the most convenient specimen will be blood samples or biopsies of suspect tissue, such as, e.g., breast, colon, kidney, liver, ovary or prostate. It is also possible and preferred in some circumstances to conduct assays on cells that are isolated under microscopic visualization. A particularly useful method is the microdissection technique described in WO 95/23960. The cells isolated by microscopic visualization can be used in any of the assays described herein including both genomic and immunological based assays.

This invention provides methods of genotyping family members in which relatives are diagnosed with cancer. Conventional methods of genotyping are provided herein.

The invention provides methods for determining the tissue of origin for a cell of interest, typically by contacting the cell with a probe comprising a tissue-specific polynucleotide sequence (i.e., a marker sequence). The amount of tissue-specific probe which hybridizes to the RNA is compared to the amount of the probe which hybridizes to RNA from a tissue where the marker is expressed (or is expressed at a significantly higher level) and with the amount of the probe which hybridizes to RNA from a tissue where the marker is not expressed (or is expressed at a significantly lower level).

The invention further provides methods for detecting whether a cell is in a cancerous state or is undergoing transformation and/or becoming malignant. The methods typically comprise contacting RNA from the cell with a probe which comprises a polynucleotide sequence associated with cancer, and determining whether the amount of the probe which hybridizes to the RNA is increased or decreased relative to the amount of the probe which hybridizes to RNA from a cell from a subject not suffering from cancer. The assays are useful for detecting cell transformation associated with, for example, different types of cancer. One can also detect cell proliferation, neoplasia and/or transformation using the methods of the invention.

The probes are capable of binding to a target nucleic acid (e.g., a nucleic acid associated with cancer development or a tissue-specific nucleic acid). By assaying for the presence or absence of the probe, one can detect the presence or absence of the target nucleic acid in a sample. Preferably, non-hybridizing probe and target nucleic acids are removed (e.g., by washing) prior to detecting the presence of the probe.

A variety of methods of specific DNA and RNA measurement using nucleic acid hybridization techniques are known to those of skill in the art (see, Sambrook, supra). For example, one method for evaluating the presence or absence of the DNA in a sample involves a Southern transfer. Briefly, the digested genomic DNA is run on agarose slab gels in buffer and transferred to membranes. Hybridization is carried out using the probes discussed above. Visualization of the hybridized portions allows the qualitative determination of the presence, alteration or absence of a tissue-specific and/or cancer-associated gene.

Similarly, a Northern transfer may be used for the detection of a tissue-specific mRNA and/or of a cancer-associated mRNA in samples of RNA from cells expressing the tissue-specific and/or cancer-associated proteins. In brief, the mRNA is isolated from a given cell sample using, for example, an acid guanidinium-phenol-chloroform extraction method. The mRNA is then electrophoresed to separate the mRNA species and the mRNA is transferred from the gel to a nitrocellulose membrane. As with the Southern blots, labeled probes are used to identify the presence or absence of the subject protein transcript. Alternatively, the amount of, for example, a tissue-specific or a cancer-associated mRNA can be analyzed in the absence of electrophoretic separation.

The selection of a nucleic acid hybridization format is not critical. A variety of nucleic acid hybridization formats are known to those skilled in the art. For example, common formats include sandwich assays and competition or displacement assays. Hybridization techniques are generally described in Hames and Higgins, “Nucleic Acid Hybridization, A Practical Approach” IRL Press (1985); Gall and Pardue, Proc. Natl. Acad. Sci. U.S.A. 63:378-383 (1969); and John et al., Nature 223:582-587 (1969).

For example, sandwich assays are commercially useful hybridization assays for detecting or isolating nucleic acids. Such assays utilize a “capture” nucleic acid covalently immobilized to a solid support and a labeled “signal” nucleic acid in solution. The clinical sample will provide the target nucleic acid. The “capture” nucleic acid and “signal” nucleic acid probe hybridize with the target nucleic acid to form a “sandwich” hybridization complex. To be effective, the signal nucleic acid cannot hybridize with the capture nucleic acid.

Detection of a hybridization complex may require the binding of a signal-generating complex to a duplex of target and probe polynucleotides or nucleic acids. Typically, such binding occurs through ligand and anti-ligand interactions as between a ligand-conjugated probe and an anti-ligand conjugated with a signal. The binding of the signal generation complex is also readily amenable to accelerations by exposure to ultrasonic energy.

The label may also allow indirect detection of the hybridization complex. For example, where the label is a hapten or antigen, the sample can be detected by using antibodies. In these systems, a signal is generated by attaching fluorescent or enzyme molecules to the antibodies or in some cases, by attachment to a radioactive label (see, e.g., Tijssen, “Practice and Theory of Enzyme Immunoassays” Laboratory Techniques in Biochemistry and Molecular Biology, Burdon and van Knippenberg Eds., Elsevier (1985), pp. 9-20).

The probes are typically labeled either directly, as with isotopes, chromophores, lumiphores, chromogens, or indirectly, such as with biotin, to which a streptavidin complex may later bind. Thus, the detectable labels used in the assays of the present invention can be primary labels (where the label comprises an element that is detected directly or that produces a directly detectable element) or secondary labels (where the detected label binds to a primary label, e.g., as is common in immunological labeling). Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P-labeled probes or the like.

Other labels include, e.g., ligands which bind to labeled antibodies, fluorophores, chemiluminescent agents, enzymes, and antibodies which can serve as specific binding pair members for a labeled ligand. An introduction to labels, labeling procedures and detection of labels is found in Polak and Van Noorden, Introduction to Immunocytochemistry, 2nd ed., Springer Verlag, NY (1997); and in Haugland Handbook of Fluorescent Probes and Research Chemicals, a combined handbook and catalogue Published by Molecular Probes, Inc. (1996). Primary and secondary labels can include undetected elements as well as detected elements. Useful primary and secondary labels in the present invention can include spectral labels such as fluorescent dyes (e.g., fluorescein and derivatives such as fluorescein isothiocyanate (FITC) and Oregon Green™, rhodamine and derivatives (e.g., Texas red, tetrarhodimine isothiocynate (TRITC), etc.), digoxigenin, biotin, phycoerythrin, AMCA, CyDyes™, and the like), radiolabels (e.g., ³H, ¹²⁵I, ³⁵S, ¹⁴C, ³²P, ³³P, etc.), enzymes (e.g., horse radish peroxidase, alkaline phosphatase, etc.), spectral calorimetric labels such as colloidal gold or colored glass or plastic (e.g., polystyrene, polypropylene, latex, etc.) beads. The label may be coupled directly or indirectly to a component of the detection assay (e.g., the probe) according to methods well known in the art. As indicated above, a wide variety of labels may be used, with the choice of label depending on the sensitivity required, the ease of conjugation with the compound, stability requirements, available instrumentation, and disposal provisions.

Preferred labels include those that use: 1) chemiluminescence (using horseradish peroxidase and/or alkaline phosphatase with substrates that produce photons as breakdown products as described above) with kits being available, e.g., from Molecular Probes, Amersham, Boehringer-Mannheim, and Life Technologies/Gibco BRL; 2) color production (using both horseradish peroxidase and/or alkaline phosphatase with substrates that produce a colored precipitate [kits available from Life Technologies/Gibco BRL, and Boehringer-Mannheim]); 3) hemifluorescence using, e.g., alkaline phosphatase and the substrate AttoPhos [Amersham] or other substrates that produce fluorescent products, 4) fluorescence (e.g., using Cy-5 [Amersham]), fluorescein, and other fluorescent tags); and 5) radioactivity. Other methods for labeling and detection will be readily apparent to one skilled in the art.

Preferred enzymes that can be conjugated to detection reagents of the invention include, e.g., β-galactosidase, luciferase, horse radish peroxidase, and alkaline phosphatase. The chemiluminescent substrate for luciferase is luciferin. One embodiment of a chemiluminescent substrate for β-galactosidase is 4-methylumbelliferyl-β-D-galactoside. Embodiments of alkaline phosphatase substrates include p-nitrophenyl phosphate (pNPP), which is detected with a spectrophotometer; 5-bromo-4-chloro-3-indolyl phosphate/nitro blue tetrazolium (BCIP/NBT) and fast red/napthol AS-TR phosphate, which are detected visually; and 4-methoxy-4-(3-phosphonophenyl) spiro[1,2-dioxetane-3,2′-adamantane], which is detected with a luminometer. Embodiments of horse radish peroxidase substrates include 2,2′azino-bis(3-ethylbenzthiazoline-6 sulfonic acid) (ABTS), 5-aminosalicylic acid (5AS), o-dianisidine, and o-phenylenediamine (OPD), which are detected with a spectrophotometer; and 3,3,5,5′-tetramethylbenzidine (TMB), 3,3′diaminobenzidine (DAB), 3-amino-9-ethylcarbazole (AEC), and 4-chloro-1-naphthol (4C1N), which are detected visually. Other suitable substrates are known to those skilled in the art. The enzyme-substrate reaction and product detection are performed according to standard procedures well known to those skilled in the art and kits for performing enzyme immunoassays are available as described herein.

In general, a detector which monitors a particular probe or probe combination is used to detect the detection reagent label. Typical detectors include spectrophotometers, phototubes and photodiodes, microscopes, scintillation counters, cameras, film and the like, as well as combinations thereof. Examples of suitable detectors are widely available from a variety of commercial sources known to persons of skill in the art. Commonly, an optical image of a substrate comprising bound labeling moieties is digitized for subsequent computer analysis.

Most typically, the amount of, for example, a tissue-specific and/or a cancer-associated RNA is measured by quantitating the amount of label fixed to the solid support by binding of the detection reagent. Typically, the presence of a modulator during incubation will increase or decrease the amount of label fixed to the solid support relative to a control incubation which does not comprise the modulator, or as compared to a baseline established for a particular reaction type. Means of detecting and quantitating labels are well known to those of skill in the art. Thus, for example, where the label is a radioactive label, means for detection include a scintillation counter or photographic film as in autoradiography. Where the label is optically detectable, typical detectors include microscopes, cameras, phototubes and photodiodes and many other detection systems which are widely available.

In preferred embodiments, the target nucleic acid or the probe is immobilized on a solid support. Solid supports suitable for use in the assays of the invention are known to those of skill in the art. As used herein, a solid support is a matrix of material in a substantially fixed arrangement. Exemplar solid supports include glasses, plastics, polymers, metals, metalloids, ceramics, organics, etc. Solid supports can be flat or planar, or can have substantially different conformations. For example, the substrate can exist as particles, beads, strands, precipitates, gels, sheets, tubing, spheres, containers, capillaries, pads, slices, films, plates, dipsticks, slides, etc. Magnetic beads or particles, such as magnetic latex beads and iron oxide particles, are examples of solid substrates that can be used in the methods of the invention. Magnetic particles are described in, for example, U.S. Pat. No. 4,672,040, and are commercially available from, for example, PerSeptive Biosystems, Inc. (Framingham Mass.), Ciba Corning (Medfield Mass.), Bangs Laboratories (Carmel Ind.), and BioQuest, Inc. (Atkinson N.H.). The substrate is chosen to maximize signal to noise ratios, primarily to minimize background binding, for ease of washing and cost.

A variety of automated solid-phase assay techniques are also appropriate. For instance, very large scale immobilized polymer arrays (VLSIPS™), available from Affymetrix, Inc. (Santa Clara, Calif.) can be used to detect changes in expression levels of a plurality of tissue-specific or cancer-associated nucleic acids simultaneously (see, Tijssen, supra; Fodor et al., Science 251:767-777 (1991); Sheldon et al., Clinical Chemistry 39(4):718-719 (1993); and Kozal et al., Nature Medicine 2(7):753-759 (1996)). Thus, in one embodiment, the invention provides methods for detecting the expression levels of tissue-specific or cancer-associated nucleic acids, in which nucleic acids (e.g., RNA from a cell culture) are hybridized to an array of nucleic acids that are known to be expressed in specific tissues and/or to be associated with cancer development. For example, in the assay described supra, oligonucleotides which hybridize to a plurality of tissue-specific (or cancer-associated) nucleic acids are optionally synthesized on a DNA chip (such chips are available from Affymetrix) and the RNA from a biological sample (e.g., from a biopsy or from a cell culture) is hybridized to the chip for simultaneous analysis of multiple tissue-specific nucleic acids (or cancer-associated nucleic acids). The tissue specific (or cancer-associated) nucleic acids that are present in the sample which is assayed are detected at specific positions on the chip.

Detection can be accomplished, for example, by using a labeled detection moiety that binds specifically to duplex nucleic acids (e.g., an antibody that is specific for RNA-DNA duplexes). One preferred example uses an antibody that recognizes DNA-RNA heteroduplexes in which the antibody is linked to an enzyme (typically by recombinant or covalent chemical bonding). The antibody is detected when the enzyme reacts with its substrate, producing a detectable product. Coutlee et al., Analytical Biochemistry 181:153-162 (1989); Bogulavski et al., J. Immunol. Methods 89:123-130 (1986); Prooijen-Knegt, Exp. Cell Res. 141:397-407 (1982); Rudkin, Nature 265:472-473 (1976); Stollar, PNAS 65:993-1000 (1970); Ballard, Mol. Immunol. 19:793-799 (1982); Pisetsky and Caster, Mol. Immunol. 19:645-650 (1982); Viscidi et al., J. Clin. Microbial. 41:199-209 (1988); and Kiney et al., J. Clin. Microbiol. 27:6-12 (1989) describe antibodies to RNA duplexes, including homo and heteroduplexes. Kits comprising antibodies specific for DNA:RNA hybrids are available, e.g., from Digene Diagnostics, Inc. (Beltsville, Md.).

In addition to available antibodies, one of skill in the art can easily make antibodies specific for nucleic acid duplexes using existing techniques, or modify those antibodies which are commercially or publicly available. In addition to the art referenced above, general methods for producing polyclonal and monoclonal antibodies are known to those of skill in the art (see, e.g., Paul (ed), Fundamental Immunology, Third Edition Raven Press, Ltd., NY (1993); Coligan, Current Protocols in Immunology Wiley/Greene, NY (1991); Harlow and Lane, Antibodies: A Laboratory Manual Cold Spring Harbor Press, NY (1989); Stites et al., (eds.) Basic and Clinical Immunology (4th ed.) Lange Medical Publications, Los Altos, Calif., and references cited therein; Goding, Monoclonal Antibodies: Principles and Practice (2d ed.) Academic Press, New York, N.Y. (1986); and Kohler and Milstein, Nature 256:495-497 (1975)). Other suitable techniques for antibody preparation include selection of libraries of recombinant antibodies in phage or similar vectors (see, Huse et al., Science 246:1275-1281 (1989); and Ward et al., Nature 341:544-546 (1989)). Specific monoclonal and polyclonal antibodies and antisera will usually bind with a K_{D} of at least about 0.1 μM, preferably at least about 0.01 μM or better, and most typically and preferably, 0.001 μM or better.

The nucleic acids used in this invention can be either positive or negative probes. Positive probes bind to their targets and the presence of duplex formation is evidence of the presence of the target. Negative probes fail to bind to the suspect target and the absence of duplex formation is evidence of the presence of the target. For example, the use of a wild type specific nucleic acid probe or PCR primers may serve as a negative probe in an assay sample where only the nucleotide sequence of interest is present.

The sensitivity of the hybridization assays may be enhanced through the use of a nucleic acid amplification system which multiplies the target nucleic acid being detected. Examples of such systems include the polymerase chain reaction (PCR) system and the ligase chain reaction (LCR) system. Other methods recently described in the art are the nucleic acid sequence based amplification (NASBAθ, Cangene, Mississauga, Ontario) and Q Beta Replicase systems. These systems can be used to directly identify mutants where the PCR or LCR primers are designed to be extended or ligated only when a selected sequence is present. Alternatively, the selected sequences can be generally amplified using, for example, nonspecific PCR primers and the amplified target region later probed for a specific sequence indicative of a mutation.

A preferred embodiment is the use of allelic specific amplifications. In the case of PCR, the amplification primers are designed to bind to a portion of, for example, a gene encoding a cancer-associated protein (or a tissue-specific gene), but the terminal base at the 3′ end is used to discriminate between the mutant and wild-type forms of the cancer-associated gene (or of the tissue-specific gene). If the terminal base matches the point mutation or the wild-type, polymerase dependent three prime extension can proceed and an amplification product is detected. This method for detecting point mutations or polymorphisms is described in detail by Sommer et al., Mayo Clin. Proc. 64:1361-1372 (1989). By using appropriate controls, one can develop a kit having both positive and negative amplification products. The products can be detected using specific probes or by simply detecting their presence or absence. A variation of the PCR method uses LCR where the point of discrimination, i.e., either the point mutation or the wild-type bases fall between the LCR oligonucleotides. The ligation of the oligonucleotides becomes the means for discriminating between the mutant and wild-type forms of, e.g., the gene encoding the cancer-associated protein.

An alternative means for determining the level of expression of the nucleic acids of the present invention is in situ hybridization. In situ hybridization assays are well known and are generally described in Angerer et al., Methods Enzymol. 152:649-660 (1987). In an in situ hybridization assay, cells, preferably human cells from a biological sample (e.g., a biopsy from tissue or organ of interest or a blood sample) or from a tissue culture, are fixed to a solid support, typically a glass slide. If DNA is to be probed, the cells are denatured with heat or alkali. The cells are then contacted with a hybridization solution at a moderate temperature to permit annealing of specific probes that are labeled. The probes are preferably labeled with radioisotopes or fluorescent reporters.

VI. Immunological Detection of the Tissue-Specific Proteins of the Invention

In addition to the detection of the expression of the gene encoding the subject protein using nucleic acid hybridization technology, one can also use immunoassays to detect the protein itself. Immunoassays can be used to qualitatively or quantitatively analyze the proteins of interest. A general overview of the applicable technology can be found in Harlow and Lane, Antibodies: A Laboratory Manual, Cold Spring Harbor Pubs., NY (1988). The following discussion is directed to methods for detecting target tissue-specific proteins, including proteins associated with cancer. Similar methods can be used to detect target proteins associated with, e.g., cell proliferation, cell transformation, neoplasia, metastasis and/or target proteins associated with different types of cancer (e.g., breast, colon, liver, kidney, ovarian, prostate, etc.).

A. Antibodies to Target Proteins

Methods for producing polyclonal and monoclonal antibodies that react specifically with a protein of interest are known to those of skill in the art (see, e.g., Coligan, supra; and Harlow and Lane, supra; Stites et al., supra and references cited therein; Goding, supra; and Kohler and Milstein, Nature 256:495-497 (1975)). Such techniques include antibody preparation by selection of antibodies from libraries of recombinant antibodies in phage or similar vectors (see, Huse et al., and Ward et al., both supra). For example, in order to produce antisera for use in an immunoassay, the protein of interest, or an antigenic fragment thereof, is isolated as described herein. For example, a recombinant protein is produced in a transformed cell line. An inbred strain of mice or rabbits is immunized with the protein using a standard adjuvant, such as Freund's adjuvant, and a standard immunization protocol. Alternatively, a synthetic peptide derived from the sequences disclosed herein and conjugated to a carrier protein can be used as an immunogen.

Polyclonal sera are collected and titered against the immunogen protein in an immunoassay such as, for example, a solid phase immunoassay with the immunogen immobilized on a solid support. Polyclonal antisera with a titer of 10⁴ or greater are selected and tested for their cross-reactivity against proteins lacking or having a different tissue-specificity (including, e.g., proteins not associated with cancer) or even other homologous proteins from other organisms, using a competitive binding immunoassay. Specific monoclonal and polyclonal antibodies and antisera will usually bind with a K_{D} of at least about 0.1 mM, more usually at least about 1 μM, preferably at least about 0.1 μM or better, and most preferably, 0.01 μM or better.

A number of proteins of the invention comprising immunogens may be used to produce antibodies specifically or selectively reactive with the proteins of interest. Recombinant protein is the preferred immunogen for the production of monoclonal or polyclonal antibodies. Naturally occurring protein may also be used either in pure or impure form. Synthetic peptides made using the protein sequences described herein may also be used as an immunogen for the production of antibodies to the protein. Recombinant proteins can be expressed in eukaryotic or prokaryotic cells and purified as generally described supra. The product is then injected into an animal capable of producing antibodies. Either monoclonal or polyclonal antibodies may be generated for subsequent use in immunoassays to measure the protein.

Methods of production of polyclonal antibodies are known to those of skill in the art. In brief, an immunogen, preferably a purified protein, is mixed with an adjuvant and animals are immunized. The animal's immune response to the immunogen preparation is monitored by taking test bleeds and determining the titer of reactivity to the tissue-specific protein of interest. When appropriately high titers of antibody to the immunogen are obtained, blood is collected from the animal and antisera are prepared. Further fractionation of the antisera to enrich for antibodies reactive to the protein can be done if desired (see, Harlow and Lane, supra).

Monoclonal antibodies may be obtained using various techniques familiar to those of skill in the art. Typically, spleen cells from an animal immunized with a desired antigen are immortalized, commonly by fusion with a myeloma cell (see, Kohler and Milstein, Eur. J. Immunol. 6:511-519 (1976)). Alternative methods of immortalization include, e.g., transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse et al., supra.

Once target protein specific antibodies are available, the protein can be measured by a variety of immunoassay methods with qualitative and quantitative results available to the clinician. For a review of immunological and immunoassay procedures in general see, Stites, supra. Moreover, the immunoassays of the present invention can be performed in any of several configurations, which are reviewed extensively in Maggio, Enzyme Immunoassay, CRC Press, Boca Raton, Fla. (1980); Tijssen, supra; and Harlow and Lane, supra.

Immunoassays to measure target proteins in a human sample may use a polyclonal antiserum which was raised to the protein partially encoded by a sequence described herein or a fragment thereof. This antiserum is selected to have low cross-reactivity against proteins having a different tissue-specificity (or lacking tissue-specificity), or against proteins not being associated with cancer, and any such cross-reactivity is removed by immunoabsorption prior to use in the immunoassay.

In order to produce antisera for use in an immunoassay, the tissue-specific or cancer-associated protein of interest or a fragment thereof, for example, is isolated as described herein. For example, recombinant protein is produced in a transformed cell line. An inbred strain of mice, such as Balb/c, is immunized with the protein or a peptide using a standard adjuvant, such as Freund's adjuvant, and a standard mouse immunization protocol. Alternatively, a synthetic peptide derived from the sequences disclosed herein and conjugated to a carrier protein can be used as an immunogen. Polyclonal sera are collected and titered against the immunogen protein in an immunoassay, such as, for example, a solid phase immunoassay with the immunogen immobilized on a solid support. Polyclonal antisera with a titer of 10⁴ or greater are selected and tested for their cross-reactivity with proteins having a different tissue-specificity (or lacking tissue-specificity) or with proteins not associated with cancer, using a competitive binding immunoassay such as the one described in Harlow and Lane, supra, at pages 570-573 and below.

B. Immunological Binding Assays

In a preferred embodiment, a protein of interest is detected and/or quantified using any of a number of well known immunological binding assays (see, e.g., U.S. Pat. Nos. 4,366,241; 4,376,110; 4,517,288; and 4,837,168). For a review of the general immunoassays, see also Asai, Methods in Cell Biology Volume 37: Antibodies in Cell Biology, Academic Press, Inc. NY (1993); and Stites & Terr, supra. Immunological binding assays (or immunoassays) typically utilize a “capture agent” to specifically bind to and often immobilize the analyte (in this case the tissue-specific protein, i.e., the marker protein, or antigenic subsequence thereof). The capture agent is a moiety that specifically binds to the analyte. In a preferred embodiment, the capture agent is an antibody that specifically binds, for example, the marker protein. The antibody (e.g., anti-marker protein antibody) may be produced by any of a number of means well known to those of skill in the art and as described above.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled marker protein or a labeled anti-marker-protein antibody. Alternatively, the labeling agent may be a third moiety, such as another antibody, that specifically binds to the antibody/protein complex.

In a preferred embodiment, the labeling agent is a second antibody bearing a label. Alternatively, the second antibody may lack a label, but it may, in turn, be bound by a labeled third antibody specific to antibodies of the species from which the second antibody is derived. The second antibody can be modified with a detectable moiety, such as biotin, to which a third labeled molecule can specifically bind, such as enzyme-labeled streptavidin.

Other proteins capable of specifically binding immunoglobulin constant regions, such as protein A or protein G, can also be used as the label agents. These proteins are normal constituents of the cell walls of streptococcal bacteria. They exhibit a strong non-immunogenic reactivity with immunoglobulin constant regions from a variety of species (see, generally, Kronval et al., J. Immunol. 111:1401-1406 (1973); and Akerstrom et al., J. Immunol. 135:2589-2542 (1985)).

Throughout the assays, incubation and/or washing steps may be required after each combination of reagents. Incubation steps can vary from about 5 seconds to several hours, preferably from about 5 minutes to about 24 hours. The incubation time will depend upon the assay format, analyte, volume of solution, concentrations, and the like. Usually, the assays will be carried out at ambient temperature, although they can be conducted over a range of temperatures, such as 10° C. to 40° C.

1. Non-Competitive Assay Formats

Immunoassays for detecting a protein of interest from tissue samples may be either competitive or noncompetitive. Noncompetitive immunoassays are assays in which the amount of captured analyte (in this case the marker protein) is directly measured. In one preferred “sandwich” assay, for example, the capture agent (e.g., anti-marker protein antibodies) can be bound directly to a solid substrate where it is immobilized. These immobilized antibodies then capture the marker protein present in the test sample. The marker protein thus immobilized is then bound by a labeling agent, such as a second anti-marker protein antibody bearing a label. Alternatively, the second antibody may lack a label, but it may, in turn, be bound by a labeled third antibody specific to antibodies of the species from which the second antibody is derived. The second can be modified with a detectable moiety, such as biotin, to which a third labeled molecule can specifically bind, such as enzyme-labeled streptavidin.

2. Competitive Assay Formats

In competitive assays, the amount of target protein (analyte) present in the sample is measured indirectly by measuring the amount of an added (exogenous) analyte (i.e., the marker protein of interest) displaced (or competed away) from a capture agent (anti-marker protein antibody) by the analyte present in the sample. In one competitive assay, a known amount of, in this case, the marker protein of interest is added to the sample and the sample is then contacted with a capture agent, in this case an antibody that specifically binds to the marker protein. The amount of marker protein bound to the antibody is inversely proportional to the concentration of marker protein present in the sample. In a particularly preferred embodiment, the antibody is immobilized on a solid substrate. The amount of the marker protein bound to the antibody may be determined either by measuring the amount of subject protein present in a marker protein/antibody complex or, alternatively, by measuring the amount of remaining uncomplexed protein. The amount of marker protein may be detected by providing a labeled marker protein molecule.

A hapten inhibition assay is another preferred competitive assay. In this assay, a known analyte, in this case the marker protein, is immobilized on a solid substrate. A known amount of anti-marker protein antibody is added to the sample, and the sample is then contacted with the immobilized target. In this case, the amount of anti-marker protein antibody bound to the immobilized marker protein is inversely proportional to the amount of marker protein present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

Immunoassays in the competitive binding format can be used for cross-reactivity determinations. For example, a protein encoded by a sequence described herein can be immobilized on a solid support. Proteins are added to the assay which compete with the binding of the antisera to the immobilized antigen. The ability of the above proteins to compete with the binding of the antisera to the immobilized protein is compared to that of the protein encoded by the sequence of interest. The percent cross-reactivity for the above proteins is calculated, using standard calculations. Those antisera with less than 10% cross-reactivity with each of the proteins listed above are selected and pooled. The cross-reacting antibodies are optionally removed from the pooled antisera by immunoabsorption with the considered proteins, e.g., distantly related homologues.

The immunoabsorbed and pooled antisera are then used in a competitive binding immunoassay as described above to compare a second protein, thought to be perhaps the protein of interest, to the immunogen protein. In order to make this comparison, the two proteins are each assayed at a wide range of concentrations and the amount of each protein required to inhibit 50% of the binding of the antisera to the immobilized protein is determined. If the amount of the second protein required is less than 10 times the amount of the protein partially encoded by a sequence herein that is required, then the second protein is said to specifically bind to an antibody generated to an immunogen consisting of the target marker protein.

3. Other Assay Formats

In a particularly preferred embodiment, Western blot (immunoblot) analysis is used to detect and quantify the presence of a tissue-specific protein in the sample. The technique generally comprises separating sample proteins by gel electrophoresis on the basis of molecular weight, transferring the separated proteins to a suitable solid support (such as, e.g., a nitrocellulose filter, a nylon filter, or a derivatized nylon filter) and incubating the sample with the antibodies that specifically bind the protein of interest. For example, the anti-tissue-specific protein antibodies specifically bind to the tissue-specific protein on the solid support. These antibodies may be directly labeled or alternatively may be subsequently detected using labeled antibodies (e.g., labeled sheep anti-mouse antibodies) that specifically bind to the antibodies against the tissue-specific protein of interest.

Other assay formats include liposome immunoassays (LIA), which use liposomes designed to bind specific molecules (e.g., antibodies) and release encapsulated reagents or markers. The released chemicals are then detected according to standard techniques (see, Monroe et al., Amer. Clin. Prod. Rev. 5:34-41 (1986)).

4. Reduction of Non-Specific Binding

One of skill in the art will appreciate that it is often desirable to use non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this involves coating the substrate with a proteinaceous composition. In particular, protein compositions, such as bovine serum albumin (BSA), nonfat powdered milk and gelatin, are widely used with powdered milk being most preferred.

5. Labels

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most labels useful in such methods can be applied to the present invention. Thus, a label is any composition detectable by, e.g., spectroscopic, photochemical, biochemical, immunochemical, electrical, optical or chemical means. Useful labels in the present invention include magnetic beads (e.g., Dynabeads™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads.

The label may be coupled directly or indirectly to the desired component of the assay according to methods well known in the art. As indicated above, a wide variety of labels may be used, with the choice of label depending on the sensitivity required, the ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Thyroxine, and cortisol can be used in conjunction with the labeled, naturally occurring anti-ligands. Alternatively, any haptenic or antigenic compound can be used in combination with an antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol (for a review of various labeling or signal producing systems which may be used, see, e.g., U.S. Pat. No. 4,391,904).

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple calorimetric labels may be detected directly by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need to be labeled and the presence of the target antibody is detected by simple visual inspection.

VII. Fusion Proteins

In another aspect, the present invention provides fusion proteins for targeting a therapeutic compound of interest to a specific tissue.

The fusion proteins of the invention typically comprise a first protein which specifically binds to a tissue-specific molecule of the invention (e.g., an antibody that specifically binds to a tissue-specific molecule selected from the group of ribonucleotides and proteins encoded by the polynucleotide sequences set forth in Table 1A and B) or a portion thereof, and a second protein which has therapeutic properties (i.e., a “therapeutic protein”). When only a portion of the protein that specifically binds to the tissue-specific molecule (i.e., the “binding protein”) is used, the portion is a fragment that retains the binding specificity for the tissue-specific molecule of interest. Fusion partners may include affinity tags to facilitate the purification. In some embodiments, the therapeutic protein of interest is a cytotoxin protein, i.e., a toxin. Toxins that can be used for therapeutic applications are well known in the art and include, but are not limited to, ricin, Diphtheria toxin and Pseudomonas toxin.

Portions of proteins may be generated by synthetic or recombinant means. Synthetic polypeptides having fewer than about 100 amino acids, and generally fewer 5 than about 50 amino acids, may be generated using techniques well known in the art. For example, such polypeptides may be synthesized using any of the commercially available solid-phase techniques, such as the Merrifield solid-phase synthesis method, where amino acids are sequentially added to a growing amino acid chain (see, Merrifield, J. Am. Chem. Soc. 85:2149-2146 (1963)). Equipment for automated synthesis of polypeptides is commercially available from suppliers such as Perkin Elmer/Applied BioSystems Division, Inc., Foster City, Calif., and may be operated according to the manufacturer's instructions. Sections of the DNA sequence encoding the binding protein of interest may also be removed using standard techniques to permit preparation of truncated polypeptides. The specific binding of a portion to the tissue-specific target protein of interest can be tested using a variety of methods such as, e.g., immunological binding assays as described supra.

The fusion proteins of the present invention may also include a linker peptide between the first and second polypeptides. A peptide linker sequence may be employed to separate, for example, the first and second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional portions on the first and second polypeptides; and (3) the lack of hydrophobic or charged residues that might react with the polypeptide functional portions. Amino acid sequences which may be usefully employed as linkers include those disclosed in Maratea et al., Gene 40:39-46 (1985); Murphy et al., Proc. Natl. Acad. Sci. USA 83:8258-8262 (1986); and in U.S. Pat. Nos. 4,935,233 and 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

Fusion proteins may generally be prepared using standard techniques, including chemical conjugation. Fusion proteins can also be expressed as recombinant proteins in an expression system, as described supra. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

The ligated DNA sequences are operably linked to suitable transcriptional or translational regulatory elements. The regulatory elements responsible for expression of DNA are located only 5′ to the DNA sequence encoding the first polypeptide. Similarly, stop codons required to end translation, and transcription termination signals are only present 3′ to the DNA sequence encoding the second polypeptide.

VIII. Chimeric Tissue-Specific Targeted Molecules

The present invention further provides chimeric tissue-specific targeted molecules that are useful for targeting compounds, in particular therapeutic compounds, to a tissue of interest.

A. Chimeric Molecules

The present invention provides chimeric molecules for targeting a particular compound (e.g., a therapeutic compound) to a tissue of interest. A molecule that specifically binds to a target tissue-specific molecule of the present invention (e.g., an antibody recognizing a tissue-specific molecule of the invention), or a fragment thereof that retains specificity for the tissue-specific molecule of the invention, is attached to a compound of interest. The tissue-specific molecule of the invention is preferably selected from the group consisting of the ribonucleotides and the proteins encoded by the polynucleotides set forth in Table 1A and B. The molecule that specifically binds to the tissue-specific molecule of interest (i.e., the “binding molecule”), or a fragment thereof, is therefore used as a targeting moiety. The compound attached to the binding molecule is selected according to the purpose of the targeted delivery.

The methods of targeted delivery described herein may be employed for a number of purposes. For instance, the delivery of pharmaceutical compounds at specific target sites may be used for, e.g., selectively killing or preventing the proliferation of cells (e.g., tumor cells, or cells infected with bacteria, parasites, viruses, etc.), hormone therapy, target imaging, cell sorting, separation schemes, etc. In addition, the methods of the present invention may have an application in gene therapy wherein DNA or specific genes may be delivered in vivo or in vitro to target cells that are deficient in that particular gene.

Alternatively, the chimeric molecules of the present invention may be used for locating, detecting, and/or quantitating specific sites in vivo and in vitro such as tumors, organs, or sites of infection. This embodiment of the invention is particularly useful in imaging systems, cell sorting techniques, and separation schemes.

A therapeutic agent may be coupled (e.g., covalently bonded) to a suitable molecule that specifically binds to a target tissue-specific molecule of the present invention either directly or indirectly (e.g., via a linker group). A direct reaction between an agent and a binding molecule is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and a binding molecule via a linker group. A linker group can function as a spacer to distance a binding molecule from an agent in order to avoid interference with binding capabilities. A linker group can also serve to increase the chemical reactivity of a substituent on an agent or a binding molecule, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional (such as those described in the catalog of the Pierce Chemical Co., Rockford, Ill.), may be employed as the linker group. Coupling may be effected, for example, through amino groups, carboxyl groups, sulfhydryl groups or oxidized carbohydrate residues. There are numerous references describing such methodology, including, e.g., U.S. Pat. No. 4,671,958.

Where a therapeutic agent is more potent when free from the binding molecule or portion thereof, it may be desirable to use a linker group which is cleavable during or upon internalization into a cell. A number of different cleavable linker groups have been described. The mechanisms for the intracellular release of an agent from these linker groups include cleavage by reduction of a disulfide bond (e.g., U.S. Pat. No. 4,489,710), by irradiation of a photolabile bond (e.g., U.S. Pat. No. 4,625,014), by hydrolysis of derivatized amino acid side chains (e.g., U.S. Pat. No. 4,638,045), by serum complement-mediated hydrolysis (e.g., U.S. Pat. No. 4,671,958), and by acid-catalyzed hydrolysis (e.g., U.S. Pat. No. 4,569,789).

It may be desirable to couple more than one agent to a binding molecule. In one embodiment, multiple molecules of an agent are coupled to one binding molecule. In another embodiment, more than one type of agent may be coupled to one binding molecule. Regardless of the particular embodiment, chimeric molecules with more than one agent may be prepared in a variety of ways. For example, more than one agent may be coupled directly to a binding molecule, or linkers that provide multiple sites for attachment can be used. Alternatively, a carrier can be used.

A carrier may bear the agents in a variety of ways, including covalent bonding either directly or via a linker group. Suitable carriers include proteins such as, e.g., albumins (e.g., U.S. Pat. No. 4,507,234), peptides and polysaccharides such as, e.g., aminodextran (e.g., U.S. Pat. No. 4,699,784). A carrier may also bear an agent by noncovalent bonding or by encapsulation, such as within a liposome vesicle (e.g., U.S. Pat. Nos. 4,429,008 and 4,873,088). Carriers specific for radionuclide agents include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562 discloses representative chelating compounds and their synthesis.

B. Targeted Liposomes

In addition to the chimeric molecules described above, targeted liposomes can also be used for targeting a particular compound (e.g., a therapeutic compound) to a tissue of interest and are embraced by the present invention.

A variety of methods are available for preparing liposomes as described in, e.g., Szoka, et al., Ann. Rev. Biophys. Bioeng. 9:467 (1980), U.S. Pat. Nos. 4,186,183; 4,217,344; 4,235,871; 4,261,975; 4,485,054; 4,501,728; 4,774,085; 4,837,028; and 4,946,787; PCT Publication No. WO 91/17424; Deamer and Bangham, Biochim. Biophys. Acta 443:629-634 (1976); Fraley et al., Proc. Natl. Acad. Sci. USA 76:3348-3352 (1979); Hope et al., Biochim. Biophys. Acta 812:55-65 (1985); Mayer et al., Biochim. Biophys. Acta 858:161-168 (1986); Williams et al., Proc. Natl. Acad. Sci. USA 85:242-246 (1988); Ostro (Ed.), Liposomes, Marcel Dekker, Inc., New York (1983), Chapter 1; and Hope et al., Chem. Phys. Lip. 40:89 (1986).

In the context of the present invention, the targeted liposomes preferably include a therapeutic agent to be delivered to the target tissue. The therapeutic agent may be entrapped in the aqueous space or in the lipid bilayer of the liposome. A variety of methods for the preparation of therapeutic liposomes are known in the art and are described, e.g., in U.S. Pat. Nos. 6,056,973 and 5,800,833.

Targeting of liposomes using a variety of targeting moieties has been previously described (see, e.g., U.S. Pat. Nos. 4,957,773; 4,603,044; and 6,056,973). In the present invention, the targeting moieties preferably comprise molecules that specifically bind to a tissue-specific molecule of the invention (e.g., antibodies that specifically bind to a tissue-specific molecule of the invention), or fragments thereof that retain the specific binding to the tissue-specific molecule of interest. Again, the tissue-specific molecule of the invention is preferably a ribonucleotide or a protein encoded by a nucleotide sequence selected from the group of sequences set forth in Table 1A and B.

Targeting mechanisms generally require that the targeting agents be positioned on the surface of the liposome in such a manner that the target moiety is available for interaction with the target (e.g., a cell surface receptor). The liposome is designed to incorporate a connector portion into the membrane at the time of liposome formation. The connector portion must have a lipophilic portion that is firmly embedded and anchored into the membrane. It must also have a hydrophilic portion that is chemically available on the aqueous surface of the liposome. The hydrophilic portion is selected so as to be chemically suitable with the targeting agent, such that the portion and agent form a stable chemical bond. Therefore, the connector portion usually extends out from the liposomal surface and is configured to correctly position the targeting agent. In some cases, it is possible to attach the target agent directly to the connector portion, but in many instances, it is more suitable to use a third molecule to act as a “molecular bridge.” The bridge links the connector portion and the target agent off of the surface of the liposome, thereby making the target agent freely available for interaction with the cellular target.

Standard methods for coupling the target agents can be used. For example, phosphatidylethanolamine, which can be activated for attachment of target agents, or derivatized lipophilic compounds, such as lipid-derivatized bleomycin, can be used. Antibody-targeted liposomes can also be constructed using, for instance, liposomes that incorporate protein A (see, Renneisen et al., J. Bio. Chem. 265:16337-16342 (1990) and Leonetti et al., Proc. Natl. Acad. Sci. USA 87:2448-2451 (1990). When proteins are used as targeting moieties, they can be attached to the liposomes via covalent bonds (see, Heath, Covalent Attachment ofproteins to Liposomes, Methods in Enzymology 149:111-119, Academic Press, Inc. (1987)).

C. Therapeutic Compounds

Compounds that can be targeted using the chimeric molecules or the targeted liposomes of the present invention include, but are not limited to, toxins and fragments thereof (e.g., ricin, abrin, Diptheria toxin, cholera toxin, gelonin, Pseudomonas exotoxin, Shigella toxin, and pokeweed antiviral protein), alkylating agents, cytotoxic agents (e.g., daunorubicin, mithramycin, doxorubicin, methotrexate, cytosine, Mitomycin C, etc.), enzymes, antibiotics, antimetabolites, anti-proliferative agents, hormones, neurotransmitters, nucleic acids, radioopaque dyes, radioactive isotopes (e.g., ²¹¹At, ²¹²Bi, ¹²³I, ¹²⁵I, ¹³¹I, ¹⁸⁶Re, ¹⁸⁸Re, ⁹⁰Y, etc.), differentiation inducers (e.g., phorbol esters and butyric acid), fluorogenic compounds, marker compounds, lectins, compounds which alter cell membrane permeability, etc. In addition, a combination of compounds may be used. Examples of therapeutic agents that are suitable for use with the chimeric molecules of the present invention are described in, e.g., U.S. Pat. Nos. 6,056,973; 4,093,607 and 5,698,195; and Goodman and Gilman, The Pharmacological Basis of Therapeutics, 8th Ed. Macmillan Publishing Col. (1990).

The above-listed therapeutic compounds are useful, e.g., for eliminating infection, eliminating or treating a target tissue, killing a subset of cells within a target tissue, etc. For example, where the therapeutic compound is a toxin or any other cytotoxic agent, the target tissue receiving the toxin or cytotoxic agent is eliminated. This is useful, for instance, for eliminating a tissue in which cancerous cells have been identified, without performing a surgical procedure. Typically, the tissues targeted with a toxin or a cytotoxic agent are non-vital tissues (e.g., prostate, ovary, etc.). In addition, some therapeutic agents may slow down or stop cell proliferation and can be used, e.g., to eliminate malignant cells within a specific tissue.

In one preferred embodiment of the present invention, the targeted compound is an anti-neoplastic drug or a cytotoxic drug.

IX. Screening for Modulators of the Cancer Development Process

The invention also provides methods for identifying compounds that modulate cancer development, e.g., loss of proliferation control, cell transformation, development of malignancy and/or metastasis. For example, the methods can identify compounds that increase or decrease the expression level of genes associated with cancer (e.g., cell proliferation, neoplasia, cell transformation, malignancy, metastatic ability, etc.) and cancer-related conditions. Although the following discussion is directed to methods for screening for modulators of cancer development, similar methods can be used to screen for modulators of cell proliferation, neoplasia, cell transformation or for modulators of different types of cancer (e.g., breast, colon, kidney, liver, ovarian, prostate, etc.).

Compounds that are identified as modulators of cancer development using the methods of the invention find use both in vitro and in vivo. For example, one can treat cell cultures with the modulators in experiments designed to determine the mechanisms by which cancer development is regulated. Compounds that increase tumor development and/or induce neoplasia are useful for extending the life of cell cultures that are used for the production of biological products such as recombinant proteins. In vivo uses of compounds that delay cancer development include, for example, delaying the loss of cell proliferation control and/or cell transformation and treating conditions associated with neoplasia, malignant tumor formation and/or metastasis.

The methods typically involve culturing a cell in the presence of a potential modulator to form a first cell culture. RNA (or cDNA) from the first cell culture is contacted with a probe which comprises a polynucleotide sequence associated with cancer (e.g., a polynucleotide sequence selected from the group of sequences set forth in Table 1B). The amount of probe which hybridizes to the RNA (or cDNA) from the first cell culture is determined. Typically, one determines whether the amount of probe which hybridizes to the RNA (or cDNA) is increased or decreased relative to the amount of probe which hybridizes to RNA (or cDNA) from a second cell culture grown in the absence of the modulator.

It may be further determined whether the modulator-induced increase or decrease in RNA (or cDNA) levels of the target sequence is correlated with any cancer-associated change in cellular phenotype. For example, a breast, colon, kidney, liver, ovarian or prostate cell population that is treated with a modulator which induces decreased expression of a gene that is normally up-regulated with cancer may be further tested for loss of control of cell proliferation, which is reflective, for example, of a neoplastic phenotype, for cell transformation or for metastatic ability.

Essentially any chemical compound can be used as a potential modulator in the assays of the invention, although most often compounds that can be dissolved in aqueous or organic (for example, DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial library containing a large number of potential therapeutic compounds (potential modulator compounds). Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175; Furka, Int. J. Pept. Prot. Res. 37:487-493 (1991); and Houghton et al., Nature 354:84-88 (1991)). Other chemistries for generating chemical diversity libraries can also be used. Such chemistries include, but are not limited to: peptoids (PCT Publication No. WO 91/19735), encoded peptides (PCT Publication WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., Proc. Natl. Acad. Sci. USA 90:6909-6913 (1993)), vinylogous polypeptides (Hagihara et al., J. Amer. Chem. Soc. 114:6568 (1992)), nonpeptidal peptidomimetics with β-D-glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see, Ausubel et al, Berger et al., and Sambrook et al., all supra), peptide nucleic acid libraries (see, e.g., U.S. Pat. No. 5,539,083), antibody libraries (see, e.g., Vaughn et al., Nature Biotechnology 14(3):309-314 (1996) and PCT/US96/10287), carbohydrate libraries (see, e.g., Liang et al., Science 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514), and the like.

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

As noted, the invention provides in vitro assays for identifying, in a high throughput format, compounds that can modulate cancer development. Control reactions that measure the transformation or malignancy level of the cell in a reaction that does not include a potential modulator are optional, as the assays are highly uniform. Such optional control reactions are appropriate and increase the reliability of the assay. Accordingly, in a preferred embodiment, the methods of the invention include such a control reaction. For each of the assay formats described, “no modulator” control reactions which do not include a modulator provide a background level of binding activity.

In some assays it will be desirable to have positive controls to ensure that the components of the assays are working properly. At least two types of positive controls are appropriate. First, a known activator of cancer development can be incubated with one sample of the assay, and the resulting increase in signal resulting from an increased expression level of a gene associated with cancer determined according to the methods herein. Second, a known inhibitor of cancer development can be added, and the resulting decrease in signal for the expression of a gene associated with cancer similarly detected. It will be appreciated that modulators can also be combined with activators or inhibitors to find modulators which inhibit the increase or decrease that is otherwise caused by the presence of the known modulator of cancer development.

In the high throughput assays of the invention, it is possible to screen up to several thousand different modulators in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (96) modulators. If 1536 well plates are used, then a single plate can easily assay from about 100 to about 1500 different compounds. It is possible to assay many different plates per day; assay screens for up to about 6,000-20,000, and even up to about 100,000 different compounds are possible using the integrated systems of the invention.

X. Gene Therapy Applications

A variety of human diseases can be treated by therapeutic approaches that involve stably introducing a gene into a human cell such that the gene is transcribed and the gene product is produced in the cell. Diseases amenable to treatment by this approach include inherited diseases, including those in which the defect is in a single gene. Gene therapy is also useful for treatment of acquired diseases and other conditions. For discussions on the application of gene therapy towards the treatment of genetic as well as acquired diseases, see, Miller, Nature 357:455-460 (1992), and Mulligan, Science 260:926-932 (1993).

A. Vectors for Gene Delivery

For delivery to a cell or organism, the nucleic acids of the invention can be incorporated into a vector. Examples of vectors suitable for such purposes include expression plasmids capable of directing the expression of the nucleic acids in the target cell. In other instances, the vector is a viral vector system wherein the nucleic acids are incorporated into a viral genome that is capable of transfecting the target cell. In a preferred embodiment, the nucleic acids can be operably linked to expression and control sequences that can direct expression of the gene in the desired target host cells. Thus, one can achieve expression of the nucleic acid under appropriate conditions in the target cell.

B. Gene Delivery Systems

Viral vector systems useful for the expression of the nucleic acids include, for example, naturally occurring or recombinant viral vector systems. Depending upon the particular application, suitable viral vectors include replication competent, replication deficient, and conditionally replicating viral vectors. For example, viral vectors can be derived from the genome of human or bovine adenoviruses, vaccinia virus, herpes virus, adeno-associated virus, minute virus of mice (MVM), HIV, sindbis virus, and retroviruses (including but not limited to Rous sarcoma virus), and MoMLV. Typically, the genes of interest are inserted into such vectors to allow the packaging of the gene construct, typically with accompanying viral DNA, followed by infection of a sensitive host cell and expression of the gene(s) of interest.

As used herein, “gene delivery system” refers to any means for the delivery of a nucleic acid of the invention to a target cell. In some embodiments of the invention, nucleic acids are conjugated to a cell receptor ligand for facilitated uptake (e.g., invagination of coated pits and internalization of the endosome) through an appropriate linking moiety, such as a DNA linking moiety (Wu et al., J. Biol. Chem. 263:14621-14624 (1988); and WO 92/06180). For example, nucleic acids can be linked through a polylysine moiety to asialo-oromucocid, which is a ligand for the asialoglycoprotein receptor of hepatocytes.

Similarly, viral envelopes used for packaging gene constructs that include the nucleic acids of the invention can be modified by the addition of receptor ligands or antibodies specific for a receptor to permit receptor-mediated endocytosis into specific cells (see, e.g., WO 93/20221; WO 93/14188; and WO 94/06923). In some embodiments of the invention, the DNA constructs of the invention are linked to viral proteins, such as adenovirus particles, to facilitate endocytosis (Curiel et al, Proc. Natl. Acad. Sci. U.S.A. 88: 8850-8854 (1991)). In other embodiments, molecular conjugates of the instant invention can include microtubule inhibitors (WO/9406922); synthetic peptides mimicking influenza virus hemagglutinin (Plank et al., J. Biol. Chem. 269:12918-12924 (1994)); and nuclear localization signals such as SV40 T antigen (WO93/19768).

Retroviral vectors are also useful for introducing the nucleic acids of the invention into target cells or organisms. Retroviral vectors are produced by genetically manipulating retroviruses. The viral genome of retroviruses is RNA. Upon infection, this genomic RNA is reverse transcribed into a DNA copy which is integrated into the chromosomal DNA of transduced cells with a high degree of stability and efficiency. The integrated DNA copy is referred to as a provirus and is inherited by daughter cells as is any other gene. The wild type retroviral genome and the proviral DNA have three genes: the gag, the pol and the env genes, which are flanked by two long terminal repeat (LTR) sequences. The gag gene encodes the internal structural (nucleocapsid) proteins; the pol gene encodes the RNA directed DNA polymerase (reverse transcriptase); and the env gene encodes viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site). See, Mulligan, In: Experimental Manipulation of Gene Expression, M. Inouye (ed), 155-173 (1983); Mann et al, Cell 33:153-159 (1983); Cone and Mulligan, Proc. Natl. Acad. Sci. U.S.A. 81:6349-6353 (1984).

The design of retroviral vectors is well known to those of ordinary skill in the art (see, e.g., Singer and Berg, supra). In brief, if the sequences necessary for encapsidation (or packaging of retroviral RNA into infectious virions) are missing from the viral genome, the result is a cis acting defect which prevents encapsidation of genomic RNA. However, the resulting mutant is still capable of directing the synthesis of all virion proteins. Retroviral genomes from which these sequences have been deleted, as well as cell lines containing the mutant genome stably integrated into the chromosome are well known in the art and are used to construct retroviral vectors. Preparation of retroviral vectors and their uses are described in many publications including European Patent Application EPA 0 178 220, U.S. Pat. No. 4,405,712, Gilboa, Biotechniques 4:504-512 (1986), Mann et al., supra, Cone and Mulligan, supra; Eglitis et al., Biotechniques 6:608-614 (1988); Miller et al., Biotechniques 7:981-990 (1989); Miller, supra; Mulligan, supra; and PCT Publication No. WO 92/07943).

The retroviral vector particles are prepared by recombinantly inserting the desired nucleotide sequence into a retrovirus vector and packaging the vector with retroviral capsid proteins by use of a packaging cell line. The resultant retroviral vector particle is incapable of replication in the host cell but is capable of integrating into the host cell genome as a proviral sequence containing the desired nucleotide sequence. As a result, the patient is capable of producing the polynucleotide or protein of interest.

Packaging cell lines that are used to prepare the retroviral vector particles are typically recombinant mammalian tissue culture cell lines that produce the necessary viral structural proteins required for packaging, but which are incapable of producing infectious virions. The defective retroviral vectors that are used, on the other hand, lack these structural genes but encode the remaining proteins necessary for packaging. To prepare a packaging cell line, one can construct an infectious clone of a desired retrovirus in which the packaging site has been deleted. Cells comprising this construct will express all structural viral proteins, but the introduced DNA will be incapable of being packaged. Alternatively, packaging cell lines can be produced by transforming a cell line with one or more expression plasmids encoding the appropriate core and envelope proteins. In these cells, the gag, pol, and env genes can be derived from the same or different retroviruses.

A number of packaging cell lines suitable for the present invention are also available in the prior art. Examples of these cell lines include Crip, GPE86, PA317 and PG13 (see, e.g., Miller et al., J. Virol. 65:2220-2224 (1991)). Examples of other packaging cell lines are described in Cone and Mulligan, supra; and in Danos and Mulligan, Proc. Natl. Acad. Sci. USA 85: 6460-6464 (1988), Eglitis et al., supra; and Miller, supra).

Packaging cell lines capable of producing retroviral vector particles with chimeric envelope proteins may be used. Alternatively, amphotropic or xenotropic envelope proteins, such as those produced by PA317 and GPX packaging cell lines may be used to package the retroviral vectors.

In some embodiments of the invention, an antisense nucleic acid is administered which hybridizes to a gene associated with cancer development or to a transcript thereof. The antisense nucleic acid can be provided as an antisense oligonucleotide (see, e.g., Murayama et al., Antisense Nucleic Acid Drug Dev. 7:109-114 (1997)). Genes encoding an antisense nucleic acid can also be provided; such genes can be introduced into cells by methods known to those of skill in the art. For example, one can introduce a gene that encodes an antisense nucleic acid in a viral vector, such as, for example, in hepatitis B virus (see, e.g., Ji et al., J. Viral Hepat. 4:167-173 (1997)), in adeno-associated virus (see, e.g., Xiao et al., Brain Res. 756:76-83 (1997)), or in other systems including, but not limited, to an HVJ (Sendai virus)-liposome gene delivery system (see, e.g., Kaneda et al., Ann. N. Y Acad. Sci. 811:299-308 (1997)), a “peptide vector” (see, e.g., Vidal et al., CR Acad. Sci III 32:279-287 (1997)), as a gene in an episomal or plasmid vector (see, e.g., Cooper et al., Proc. Natl. Acad. Sci. U.S.A. 94:6450-6455 (1997), Yew et al., Hum Gene Ther. 8:575-584 (1997)), as a gene in a peptide-DNA aggregate (see, e.g., Niidome et al., J. Biol. Chem. 272:15307-15312 (1997)), as “naked DNA” (see, e.g., U.S. Pat. Nos. 5,580,859 and 5,589,466), in lipidic vector systems (see, e.g., Lee et al., Crit Rev Ther Drug Carrier Syst. 14:173-206 (1997)), polymer coated liposomes (U.S. Pat. Nos. 5,213,804 and 5,013,556), cationic liposomes (U.S. Pat. Nos. 5,283,185; 5,578,475; 5,279,833; and 5,334,761), gas filled microspheres (U.S. Pat. No. 5,542,935), ligand-targeted encapsulated macromolecules (U.S. Pat. Nos. 5,108,921; 5,521,291; 5,554,386; and 5,166,320).

The polynucleotide sequences of the present invention (i.e., the polynucleotide sequences set forth in Table 1A and B, and in particular in Table 1A) can also be used to induce the differentiation of a cell into a cell having a tissue-identity of interest. Some tissue-specific molecules are indeed essential for specifying the particular identity of a tissue. Such tissue-specific molecules can be expressed in cells, preferably in undifferentiated cells (e.g., in embryonic stem cells), to induce the differentiation of the undifferentiated host cells into cells having the identity of the tissue from which the molecule is derived. This can be useful, e.g., to obtain cell cultures of a particular cell type.

C. Pharmaceutical Formulations

When used for pharmaceutical purposes, the vectors used for gene therapy are formulated in a suitable buffer, which can be any pharmaceutically acceptable buffer, such as phosphate buffered saline or sodium phosphate/sodium sulfate, Tris buffer, glycine buffer, sterile water, and other buffers known to the ordinarily skilled artisan such as those described by Good et al., Biochemistry 5:467 (1966).

The compositions can additionally include a stabilizer, enhancer or other pharmaceutically acceptable carriers or vehicles. A pharmaceutically acceptable carrier can contain a physiologically acceptable compound that acts, for example, to stabilize the nucleic acids of the invention and any associated vector. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. Examples of carriers, stabilizers or adjuvants can be found in Remington, Pharmaceutical Sciences, 15th Ed., Mack Publ. Co., Easton, Pa. (1975).

D. Administration of Formulations

The formulations of the invention can be delivered to any tissue or organ using any delivery method known to the ordinarily skilled artisan. In some embodiments of the invention, the nucleic acids of the invention are formulated in mucosal, topical, and/or buccal formulations, particularly mucoadhesive gel and topical gel formulations. Exemplary permeation enhancing compositions, polymer matrices, and mucoadhesive gel preparations for transdermal delivery are disclosed in, e.g., U.S. Pat. No. 5,346,701. In some embodiments of the invention, a therapeutic agent is formulated in ophthalmic formulations for administration to the eye.

E. Methods of Treatment

The gene therapy formulations of the invention are typically administered to a cell. The cell can be provided as part of a tissue, such as an epithelial membrane, or as an isolated cell, such as in tissue culture. The cell can be provided in vivo, ex vivo, or in vitro.

The formulations can be introduced into the tissue of interest in vivo or ex vivo by a variety of methods. In some embodiments of the invention, the nucleic acids of the invention are introduced to cells by such methods as microinjection, calcium phosphate precipitation, liposome fusion, or biolistics. In further embodiments, the nucleic acids are taken up directly by the tissue of interest.

In some embodiments of the invention, the nucleic acids of the invention are administered ex vivo to cells or tissues explanted from a patient, then returned to the patient. Examples of ex vivo administration of therapeutic gene constructs include Arteaga et al., Cancer Research 56(5):1098-1103 (1996); Nolta et al., Proc Natl. Acad. Sci. USA 93(6):2414-9 (1996); Koc et al., Seminars in Oncology 23(1):46-65 (1996); Raper et al., Annals of Surgery 223(2): 116-26 (1996); Dalesandro et al., J. Thorac. Cardi. Surg. 11(2):416-22 (1996); and Makarov et al, Proc. Natl. Acad. Sci. USA 93(1):402-6 (1996).

XI. Administration and Pharmaceutical Compositions

The polynucleotides, proteins, antibodies, modulators, targeted therapeutic compounds and targeted therapeutic liposomes of the invention can be administered directly to a subject. Compositions comprising such compounds are useful for treating a particular condition or for treating or eliminating a particular tissue in vivo. Administration is by any of the routes normally used for introducing a therapeutic compound into contact with the tissue to be treated and well-known to those of skill in the art. Although more than one route can be used to administer a particular composition, a particular route can often provide a more immediate and more effective reaction than another route.

The pharmaceutical compositions of the invention may comprise a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington, Pharmaceutical Sciences, 17^{th} ed. (1985)).

The compounds of the invention, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, orally, nasally, topically, intravenously, intraperitoneally, or intrathecally. The formulations of compounds can be presented in unit-dose or multi-dose sealed containers, such as ampoules and vials. Solutions and suspensions can be prepared from sterile powders, granules, and tablets of the kind previously described. The therapeutic compounds can also be administered as part a of prepared food or drug.

The dose administered to a patient, in the context of the present invention should be sufficient to effect a beneficial response in the subject over time. The dose will be determined by the efficacy of the particular compounds employed and the condition of the subject, as well as the body weight or surface of the area to be treated. The size of the dose also will be determined by the existence, nature, and extent of any adverse side-effects that accompany the administration of a particular compound or vector in a particular subject.

In determining the effective amount of the compound to be administered a physician may evaluate circulating plasma levels of the compound, compound toxicity, and the production of anti-compound antibodies. In general, the dose equivalent of a therapeutic compound is from about 1 ng/kg to 10 mg/kg for a typical subject.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

Table 1 below indicates genes by identification in the “LifeSpan Cluster Name” column that demonstrate tissue-specific expression. “LifeSpan ID” refers to the clone identification number in the LifeSpan collection of clusters. “Image Clone ID” refers to the clone identification number in the IMAGE Consortium library (http://image.llnl.gov). Table 1 is divided into two parts (A and B). Table 1A indicates genes that demonstrate tissue-specific expression in healthy tissues. The tissue where a given gene is specifically expressed or is expressed at a significantly higher level than in any other tissue is indicated in the “Specific Tissue” column. Table 1B indicates genes that are specifically expressed or that are expressed at significantly higher levels in cancerous tissues than in the corresponding healthy or non-cancerous tissues. The cancerous tissue where a given gene is expressed is indicated in the “Specific Tissue” column.

TABLE 1
Tissue Restricted Clone List

| | | | |
|---|---|---|---|
| LifeSpan ID | LifeSpan Cluster Name | Image Clone ID | Specific Tissue |

A. Healthy tissues

| | | | |
|---|---|---|---|
| 3961 | PROTEIN ARGININE N-METHYLTRANSFERASE 1 | 128801 | adrenal |
| 4554 | SUCCINATE DEHYDROGENASE (UBIQUINONE) FLAVOPROTEIN | 71494 | adrenal |
| | SUBUNIT | | |
| 4885 | TROPOMODULIN | 428146 | adrenal |
| 6940 | INTERLEUKIN-15 RECEPTOR ALPHA CHAIN PRECURSOR (IL15RA) | 23888 | adrenal |
| 10066 | ZINC FINGER PROTEIN | 322237 | adrenal |
| 1947 | GROWTH ARREST AND DNA-DAMAGE-INDUCIBLE PROTEIN | 427872 | brain |
| | GADD153 | | |
| 4470 | SODIUM/POTASSIUM-TRANSPORTING ATPASE ALPHA-2 CHAIN | 41233 | brain |
| 4519 | SPLICING FACTOR SC35 | 73074 | brain |
| 4557 | SUCCINYL-COA LIGASE | 193778 | brain |
| 8907 | KIAA0205 | 27309 | brain |
| 16381 | CLONE 23760 | 23760 | brain |
| 26115 | ESTS, HIGHLY SIMILAR TO NK-TUMOR RECOGNITION PROTEIN | 187044 | brain |
| | (HOMO SAPIENS) | | |
| 123495 | 1 RECORD MOVED FROM CLUSTER 21727 | 120864 | brain |
| 138696 | 1 RECORD MOVED FROM CLUSTER 56419 | 206072 | brain |
| 139013 | 1 RECORD MOVED FROM CLUSTER 56786 | 382694 | brain |
| 4586 | SYNAPTOTAGMIN V | 174520 | breast tissue |
| 8188 | HEPG2 3′ REGION MBOI CDNA, CLONE HMD4H04M3 | 269940 | breast tissue |
| 10675 | XERODERMA PIGMENTOSUM GROUP E UV-DAMAGED DNA | 220559 | breast tissue |
| | BINDING FACTOR | | |
| 12385 | CAMP-DEPENDENT PROTEIN KINASE TYPE I-ALPHA REGULATORY | 590277 | breast tissue |
| | CHAIN | | |
| 4391 | SH3 DOMAIN-CONTAINING PROTEIN SH3P17 | 469611 | cervix |
| 8330 | PROFILIN I | 155724 | cervix |
| 17843 | ESTS | 28140 | cervix |
| 18119 | ESTS, HIGHLY SIMILAR TO 60S RIBOSOMAL PROTEIN L30A | 124560 | cervix |
| | (SACCHAROMYCES CEREVISIAE) | | |
| 20077 | ESTS | 38400 | cervix |
| 30541 | VACUOLAR ATP SYNTHASE SUBUNIT B, BRAIN ISOFORM | 126446 | cervix |
| 116228 | 291 RECORDS MOVED FROM CLUSTER 16087 | 727210 | cervix |
| 499 | APOPTOSIS REGULATOR BCL-2 | 147002 | corpus |
| | | | cavernosum |
| 2059 | HEPARIN-BINDING EGF-LIKE GROWTH FACTOR | 470706 | corpus |
| | | | cavernosum |
| 461 | ANNEXIN VII | 503178 | gallbladder |
| 3540 | ONCONEURAL VENTRAL ANTIGEN-1 | 37855 | gallbladder |
| 4770 | TRANSCRIPTION FACTOR E2F1 | 345890 | gallbladder |
| 39515 | ESTS, HIGHLY SIMILAR TO COFILIN, MUSCLE ISOFORM (MUS | 488362 | gallbladder |
| | MUSCULUS) | | |
| 57036 | | 546995 | gallbladder |
| 118235 | 1 RECORD MOVED FROM CLUSTER 18089 | 588368 | gallbladder |
| 57 | 26S PROTEASOME SUBUNIT 9 | 566474 | heart |
| 155 | 60 KD RO PROTEIN | 252923 | heart |
| 301 | ADP-RIBOSYLATION FACTOR 5 | 205896 | heart |
| 468 | ANTIGEN KI-67 | 120972 | heart |
| 508 | AQUAPORIN-CHIP | 628768 | heart |
| 558 | SYNTHASE LIPID-BINDING PROTEIN P2 | 182201 | heart |
| 851 | CARBOXYPEPTIDASE Z | 724310 | heart |
| 927 | CDM PROTEIN | 111265 | heart |
| 1045 | COLLAGEN ALPHA 1 (XVI) CHAIN | 255836 | heart |
| 1225 | CYTOCHROME P450-CC24 MITOCHONDRIAL | 266146 | heart |
| 1444 | EARLY GROWTH RESPONSE PROTEIN 1 | 68041 | heart |
| 1464 | ELECTRON TRANSFER FLAVOPROTEIN ALPHA-SUBUNIT | 512008 | heart |
| 1479 | ENDOGLIN | 44883 | heart |
| 1482 | ENDOPLASMIC RETICULUM PROTEIN ERP28 | 531468 | heart |
| 1885 | GLUTATHIONE SYNTHETASE | 69075 | heart |
| 1981 | GUANINE NUCLEOTIDE-BINDING PROTEIN G(I), ALPHA-1 SUBUNIT | 323694 | heart |
| 2998 | MACROPHAGE COLONY STIMULATING FACTOR-1 | 755654 | heart |
| 3197 | MONOCYTE CHEMOTACTIC PROTEIN 3 | 32873 | heart |
| 3207 | MPV 17 PROTEIN | 125302 | heart |
| 3235 | MYC-ASSOCIATED ZINC FINGER PROTEIN | 127180 | heart |
| 3304 | N-CHIMAERIN | 565488 | heart |
| 3433 | NHP2/RS6 FAMILY PROTEIN YEL026W HOMOLOG | 470772 | heart |
| 3440 | NIP2 | 563379 | heart |
| 3441 | NITRIC OXIDE SYNTHASE, INDUCIBLE | 238832 | heart |
| 3544 | G protein-coupled receptor KIAA0001 | 136983 | heart |
| 3685 | PHOSPHATIDYLINOSITOL 3-KINASE | 549264 | heart |
| 3753 | ALKALINE PHOSPHODIESTERASE I | 123269 | heart |
| 3889 | PROCHOLECYSTOKININ | 178091 | heart |
| 3895 | PROCOLLAGEN ALPHA 2 (I) CHAIN | 414994 | heart |
| 4270 | RIBOSOMAL PROTEIN S6 KINASE 2 | 490457 | heart |
| 4271 | RIBOSOMAL PROTEIN S6 KINASE II ALPHA 2 | 198833 | heart |
| 4280 | PHOSPHODIESTERASE PDE6A | 275571 | heart |
| 4342 | SERINE/THREONINE PROTEIN KINASE RIP | 592125 | heart |
| 4594 | SYNTAXIN 4 | 511867 | heart |
| 4654 | TAT-SF1 | 564812 | heart |
| 4657 | TBX5 PROTEIN | 301070 | heart |
| 4682 | THIOL-SPECIFIC ANTIOXIDANT | 755532 | heart |
| 4778 | TRANSCRIPTION FACTOR HTF4 | 261836 | heart |
| 5103 | VALYL-TRNA SYNTHETASE | 78269 | heart |
| 4558 | KIAA0379 | 25428 | heart |
| 5508 | PROTEIN KINASE PKU-ALPHA | 565171 | heart |
| 5577 | C-MYC BINDING PROTEIN | 197336 | heart |
| 6001 | NUCLEAR FACTOR NF90 | 782154 | heart |
| 7518 | NADH-UBIQUINONE OXIDOREDUCTASE CHAIN 6 | 781528 | heart |
| 15219 | C-1 | 320143 | heart |
| 19011 | ESTS, WEAKLY SIMILAR TO GP330 | 162680 | heart |
| 19554 | BRANCHED CHAIN ACYL-COA OXIDASE | 234093 | heart |
| 20871 | CHROMOSOME 17, CLONE 297N7 | 125224 | heart |
| 25960 | ESTS, HIGHLY SIMILAR TO GLUTAMATE RECEPTOR 4 PRECURSOR | 177844 | heart |
| | (RATTUS NORVEGICUS) | | |
| 30132 | CHLORIDE INTRACELLULAR CHANNEL PROTEIN 2 (CLIC2) | 305790 | heart |
| 30170 | ESTS, HIGHLY SIMILAR TO HYPOTHETICAL 33.8 KD PROTEIN | 305967 | heart |
| | R10E11.4 IN CHROMOSOME III (CAENORHABDITIS ELEGANS) | | |
| 36586 | ESTS, MODERATELY SIMILAR TO PROTEIN TRANSLATION FACTOR | 758126 | heart |
| | SUI1 HOMOLOG (MUS MUSCULUS) | | |
| 56151 | | 120177 | heart |
| 57023 | MOUSE FACTOR H | 546492 | heart |
| 57177 | P18 | 713005 | heart |
| 58843 | | 127049 | heart |
| 119565 | 26 RECORDS MOVED FROM CLUSTER 18987 | 27494 | heart |
| 128317 | 1 RECORD MOVED FROM CLUSTER 25599 | 151231 | heart |
| 138491 | 1 RECORD MOVED FROM CLUSTER 56172 | 123618 | heart |
| 138600 | 1 RECORD MOVED FROM CLUSTER 56275 | 155017 | heart |
| 138656 | 1 RECORD MOVED FROM CLUSTER 56368 | 193707 | heart |
| 157035 | 1 RECORD MOVED FROM CLUSTER 1895 | 243928 | heart |
| 1899 | GLYCINE RECEPTOR BETA CHAIN | 28471 | jejunum |
| 8958 | KIAA0262 | 46954 | jejunum |
| 25027 | ESTS | 51186 | jejunum |
| 896 | CCAAT DISPLACEMENT PROTEIN | 25304 | kidney |
| 4129 | RAB GDP DISSOCIATION INHIBITOR ALPHA | 525528 | kidney |
| 8980 | KIAA0244 | 141631 | kidney |
| 80 | RIBOSOMAL PROTEIN S11 | 78084 | Liver |
| 126 | 5-FORMYLTETRAHYDROFOLATE CYCLO-LIGASE | 78604 | Liver |
| 149 | 6-PHOSPHOFRUCTOKINASE, LIVER TYPE | 727634 | Liver |
| 409 | ALPHA-SYNUCLEIN | 193174 | Liver |
| 449 | ANGIOTENSINOGEN | 78250 | Liver |
| 474 | ANTITHROMBIN-III | 85643 | Liver |
| 748 | C-REACTIVE PROTEIN | 78282 | Liver |
| 801 | CALPAIN 1, LARGE | 84298 | Liver |
| 1052 | COLLAGEN ALPHA 4 (IV) CHAIN | 229692 | Liver |
| 1074 | COMPLEMENT COMPONENT C9 | 83153 | Liver |
| 1076 | COMPLEMENT FACTOR B | 78318 | Liver |
| 1197 | CYTOCHROME P450 IIA7 | 78298 | Liver |
| 1207 | CYTOCHROME P450 IIE1 | 77826 | Liver |
| 1434 | DYNEIN LIGHT CHAIN 1, CYTOPLASMIC | 200585 | Liver |
| 1465 | ELECTRON TRANSFER FLAVOPROTEIN BETA-SUBUNIT | 85609 | Liver |
| 1506 | EPHRIN TYPE-A RECEPTOR 1 | 85953 | Liver |
| 1893 | GLYCINE AMIDINOTRANSFERASE | 74021 | Liver |
| 2360 | HSC70-INTERACTING PROTEIN | 74667 | Liver |
| 2704 | INTERFERON-INDUCED 17KDA MEMBRANE PROTEIN | 84178 | Liver |
| 3246 | MYELOBLAST KIAA0223 | 484504 | Liver |
| 3458 | NONSPECIFIC LIPID-TRANSFER PROTEIN; sterol carrier protein X/sterol | 61643 | Liver |
| | carrier protein 2 | | |
| 3636 | PARATHYMOSIN | 82843 | Liver |
| 3750 | PLASMA PROTEASE C1 INHIBITOR | 78055 | Liver |
| 3876 | DIHYDRODIOL DEHYDROGENASE | 83310 | Liver |
| 3913 | PROLINE-RICH PROTEIN PRCC | 360164 | Liver |
| 4193 | REGULATOR OF CHROMOSOME CONDENSATION | 110652 | Liver |
| 5021 | UDP-GLUCURONOSYLTRANSFERASE 1-2 PRECURSOR, | 511275 | Liver |
| | MICROSOMAL | | |
| 5028 | UDP-GLUCURONOSYLTRANSFERASE 2B11 PRECURSOR, | 78704 | Liver |
| | MICROSOMAL | | |
| 5134 | VITAMIN D-BINDING PROTEIN | 78013 | Liver |
| 7544 | KIAA0183 | 78252 | Liver |
| 8527 | ORIP BINDING PROTEIN (OBP-2) | 210713 | Liver |
| 11020 | ENDOGENOUS RETROVIRUS TYPE C | 415562 | Liver |
| 12892 | INSULIN RECEPTOR | 427812 | Liver |
| 14116 | SERINE KINASE SRPK1 | 246240 | Liver |
| 19575 | CARBONYL REDUCTASE | 82627 | Liver |
| 24462 | ESTS, WEAKLY SIMILAR TO HYPOTHETICAL PROTEIN | 429242 | Liver |
| 27737 | ALPHA-1B-GLYCOPROTEIN | 85531 | Liver |
| 56951 | GLUTATHIONE-S TRANSFERASE HOMOLOG | 530824 | Liver |
| 57265 | | 85164 | Liver |
| 59894 | | 80765 | Liver |
| 86978 | FIBRINOGEN BETA CHAIN | 83050 | Liver |
| 116797 | 1 RECORD MOVED FROM CLUSTER 16392 | 276469 | Liver |
| 120610 | 3 RECORDS MOVED FROM CLUSTER 19725 | 772218 | Liver |
| 120840 | 3 RECORDS MOVED FROM CLUSTER 19982 | 82322 | Liver |
| 122093 | 1 RECORD MOVED FROM CLUSTER 20688 | 121628 | Liver |
| 138820 | 1 RECORD MOVED FROM CLUSTER 56559 | 25656 | Liver |
| 138959 | 1 RECORD MOVED FROM CLUSTER 56691 | 32212 | Liver |
| 143357 | 92 RECORDS MOVED FROM CLUSTER 142890 | 85642 | Liver |
| 147557 | 1 RECORD MOVED FROM CLUSTER 33727 | 627105 | Liver |
| 151912 | CYTOCHROME P450 IIC8 | 85153 | Liver |
| 152345 | 735 RECORDS MOVED FROM CLUSTER 151806 | 62173 | Liver |
| 183092 | 1 RECORD MOVED FROM CLUSTER 12405 | 288695 | Liver |
| 565 | ATP-DEPENDENT DNA HELICASE Q1 | 119808 | Lung |
| 34937 | ESTS, WEAKLY SIMILAR TO NADH-UBIQUINONE | 142144 | Lung |
| | OXIDOREDUCTASE CHAIN 5 (ASCARIS SUUM) | | |
| 135825 | 1 RECORD MOVED FROM CLUSTER 44497 | 238349 | Lung |
| 223 | ABBP-1 | 428593 | Muscle |
| 230 | ACETYLCHOLINE RECEPTOR, BETA-1 CHAIN | 612253 | Muscle |
| 292 | ADP, ATP CARRIER PROTEIN, FIBROBLAST ISOFORM | 469990 | Muscle |
| 411 | ALPHA-TRYPTASE | 183599 | Muscle |
| 577 | ATROPHIN-1 | 29510 | Muscle |
| 659 | BETA-HEXOSAMINIDASE ALPHA CHAIN | 501874 | Muscle |
| 858 | CARNITINE O-ACETYLTRANSFERASE | 485886 | Muscle |
| 1016 | CMP-N ACETYLNEURAMINATE-BETA-GALACTOSMIDE-ALPHA-2,6- | 115336 | Muscle |
| | SIALYLTRANSFERASE | | |
| 1304 | DIHYDROPYRIDINE-SENSITIVE L-TYPE, SKELETAL MUSCLE | 628206 | Muscle |
| | CALCIUM CHANNEL | | |
| 1617 | FARNESYL-DIPHOSPHATE FARNESYLTRANSFERASE | 208187 | Muscle |
| 2062 | HEPATITIS C-ASSOCIATED MICROTUBULAR AGGREGATE PROTEIN | 612217 | Muscle |
| | P44 | | |
| 2347 | HOMEOBOX PROTEIN SIX1 | 611716 | Muscle |
| 2367 | HUR RNA BINDING PROTEIN | 632302 | Muscle |
| 3050 | MD-7 PROTEIN | 267158 | Muscle |
| 3052 | MEGAKARYOCYTE GROWTH AND DEVELOPMENT FACTOR | 666797 | Muscle |
| 3438 | NIL-2-A ZINC FINGER PROTEIN | 471218 | Muscle |
| 3838 | PROTEIN KINASE PRK2 | 550355 | Muscle |
| 4416 | SKELETAL MUSCLE LIM-PROTEIN 2 | 628613 | Muscle |
| 5241 | ZINC FINGER PROTEIN 26 | 201502 | Muscle |
| 15747 | SER-THR PROTEIN KINASE PK428 | 302177 | Muscle |
| 18906 | ESTS, HIGHLY SIMILAR TO MELANOMA-ASSOCIATED ANTIGEN XP | 300080 | Muscle |
| | (HOMO SAPIENS) | | |
| 33232 | ESTS | 725558 | Muscle |
| 41450 | SMALL PROLINE RICH PROTEIN II (P22532) | 729942 | Muscle |
| 45297 | ESTS | 51930 | Muscle |
| 56624 | ORNITHINE AMINOTRANSFERASE | 289558 | Muscle |
| 130458 | 4 RECORDS MOVED FROM CLUSTER 27973 | 344618 | Muscle |
| 479 | APK1 ANTIGEN | 488063 | Pancreas |
| 14355 | ACIDIC 82 KDA PROTEIN | 328478 | Pancreas |
| 19332 | ESTS, HIGHLY SIMILAR TO MANNOSYL-OLIGOSACCHARIDE | 130586 | Pancreas |
| | ALPHA-1, 2-MANNOSIDASE (MUS MUSCULUS) | | |
| 499 | APOPTOSIS REGULATOR BCL-2 | 259606 | skin |
| 1285 | DESMOPLAKIN I | 365757 | skin |
| 26146 | ESTS, WEAKLY SIMILAR TO TWIST RELATED PROTEIN (XENOPUS | 162198 | skin |
| | LAEVIS) | | |
| 11193 | MYELIN PROTEOLIPID PROTEIN | 27308 | Testis |
| 217 | 9 KD PROTEIN | 298461 | Thyroid |
| 1606 | EXTRACELLULAR SUPEROXIDE DISMUTASE | 80774 | Thyroid |
| 3150 | MICROSOMAL TRIGLYCERIDE TRANSFER PROTEIN, LARGE | 418090 | Thyroid |
| | SUBUNIT | | |
| 11638 | DNA/ENDOGENOUS RETROVIRAL LONG TERMINAL REPEAT (LTR) | 743203 | Thyroid |
| | JUNCTION | | |
| 17931 | ESTS | 358097 | Thyroid |
| 21661 | ESTS, WEAKLY SIMILAR TO CYTOSKELETON-LIKE BICAUDAL D | 302266 | Thyroid |
| | PROTEIN (DROSOPHILA MELANOGASTER) | | |
| 59344 | | 321400 | Thyroid |
| 150827 | 29 RECORDS MOVED FROM CLUSTER 149485 | 195977 | Thyroid |
| 4500 | SPECTRIN BETA CHAIN, BRAIN | 291187 | Trachea |
| 33316 | PROTEASE INHIBITOR 12 (PI12; NEUROSERPIN) | 46040 | Trachea |
| 952 | CENTROMERE PROTEIN C | 503181 | Uterus |
| 1287 | DESTRIN | 152271 | Uterus |
| 3085 | MEMBRANE TRANSPORT PROTEIN XK | 51599 | Uterus |
| 3609 | RNA-DEPENDENT HELICASE P68 | 360029 | Uterus |
| 4081 | PUROMYCIN-SENSITIVE AMINOPEPTIDASE | 486861 | Uterus |
| 4189 | RECOVERIN | 189981 | Uterus |
| 4367 | SERINE/THREONINE PROTEIN KINASE RECEPTOR R3 | 160146 | Uterus |
| 4974 | U1 SMALL NUCLEAR RIBONUCLEOPROTEIN C | 624509 | Uterus |
| 19503 | ESTS, WEAKLY SIMILAR TO SEMAPHORIN C (M. MUSCULUS) | 293309 | Uterus |
| 33622 | ESTS, HIGHLY SIMILAR TO ATP SYNTHASE EPSILON CHAIN, | 471199 | Uterus |
| | MITOCHONDRIAL PRECURSOR (BOS TAURUS) | | |
| 139277 | 1 RECORD MOVED FROM CLUSTER 57192 | 727477 | Uterus |

B: Cancerous tissues

| | | | |
|---|---|---|---|
| 332 | ALCOHOL DEHYDROGENASE CLASS II PI CHAIN | 429323 | breast cancer |
| 424 | AMILORIDE-SENSITIVE SODIUM CHANNEL BETA-SUBUNIT | 163045 | breast cancer |
| 1526 | EPIDERMAL GROWTH FACTOR RECEPTOR | 60493 | breast cancer |
| 2055 | HEP27 PROTEIN | 188233 | breast cancer |
| 2712 | INTERLEUKIN-1 BETA CONVERTASE | 120106 | breast cancer |
| 3595 | P2Y purinoceptor 1 | 80357 | breast cancer |
| 4438 | SMOOTH MUSCLE PROTEIN 22-ALPHA | 174538 | breast cancer |
| 4684 | THIOREDOXIN REDUCTASE | 269249 | breast cancer |
| 4688 | THROMBOMODULIN | 341244 | breast cancer |
| 25876 | ESTS | 159484 | breast cancer |
| 2636 | INSULIN-LIKE GROWTH FACTOR BINDING PROTEIN 5 | 38481 | colon cancer |
| 2778 | JUNCTION PLAKOGLOBIN | 126320 | colon cancer |
| 39115 | ESTS, HIGHLY SIMILAR TO COP1 REGULATORY PROTEIN | 471128 | colon cancer |
| | (ARABIDOPSIS THALIANA) | | |
| 1799 | GAP JUNCTION ALPHA-4 PROTEIN | 153577 | Kidney Cancer |
| 1840 | GLUCOSE TRANSPORTER TYPE 1, ERYTHROCYTE/BRAIN | 347397 | Kidney Cancer |
| 4227 | RETINOBLASTOMA-LIKE PROTEIN 2 | 270456 | Kidney Cancer |
| 28770 | NITRIC-OXIDE SYNTHASE, BRAIN | 277404 | Kidney Cancer |
| 44823 | ESTS, HIGHLY SIMILAR TO (S)-2-HYDROXY-ACID OXIDASE, | 415571 | Kidney Cancer |
| | PEROXISOMAL (SPINACIA OLERACEA) | | |
| 138798 | 1 RECORD MOVED FROM CLUSTER 56535 | 248490 | Kidney Cancer |
| 1222 | CYTOCHROME P450 XIXA1 | 233941 | Liver Cancer |
| 2373 | HYDROXYMETHYLGLUTARYL-COA SYNTHASE, | 243280 | Liver Cancer |
| | MITOCHONDRIAL | | |
| 2868 | L-PLASTIN | 297058 | Liver Cancer |
| 223 | ABBP-1 | 418265 | Ovarian Cancer |
| 3725 | PHOSPHOTYROSYL PHOSPHATASE ACTIVATOR | 742063 | Ovarian Cancer |
| 4394 | SHB | 724911 | Ovarian Cancer |
| 4717 | THYROID RECEPTOR INTERACTING PROTEIN 11 | 470234 | Ovarian Cancer |
| 4807 | TRANSCRIPTION INITIATION ACTOR IIE, ALPHA SUBUNIT | 760220 | Ovarian Cancer |
| 4993 | UBIQUITIN CARBOXYL-TERMINAL HYDROLASE UHX1 | 132770 | Ovarian Cancer |
| 8461 | DOC2 (DOUBLE C2) | 26548 | Ovarian Cancer |
| 31737 | ESTS | 525465 | Ovarian Cancer |
| 37425 | ESTS | 69940 | Ovarian Cancer |
| 56983 | ESCHERICHIA COLI GENOMIC DNA. (21.3-21.7 MIN) | 544554 | Ovarian Cancer |
| 148742 | ORPHAN PROTEIN KINASE | 714116 | Ovarian Cancer |
| 133137 | ORPHAN PROTEIN KINASE | 510145 | Prostate Cancer |

What is claimed is:

1. A method for expressing a molecule in a tissue of interest, said method comprising operably linking a polynucleotide encoding said molecule to a tissue-specific promoter, wherein said tissue-specific promoter is the promoter of a gene selected from the genes set forth in Table 1A and B.

2. The method of claim 1, wherein said molecule is a protein.

3. The method of claim 1, wherein said molecule is a nucleic acid.

4. A method for diagnosing cancer in a subject, said method comprising the step of detecting the expression of a cancer-associated molecule according to Table 1B in a tissue of interest in said subject, wherein expression of said molecule is indicative of cancer of the tissue of interest.

5. The method of claim 4, comprising detecting an mRNA encoding said cancer-associated molecule.

6. The method of claim 4, comprising detecting said cancer-associated molecule in an immunoassay.

7. A method for determining the tissue of origin for a cell, said method comprising the step of detecting the expression in said cell of a tissue-specific molecule selected from the group consisting of the molecules set forth in Table 1A and B.

8. The method of claim 7, comprising detecting an mRNA encoding said tissue-specific molecule.

9. The method of claim 7, comprising detecting said tissue-specific molecule in an immunoassay.

10. The method of claim 7, wherein said cell is a cancerous cell.

11. A fusion protein comprising:
(a) at least a portion of a protein, wherein said portion specifically binds to a target protein, wherein said target protein is selected from the group consisting of the proteins encoded by the polynucleotides set forth in Table 1A and B; and
(b) a second, heterologous protein.

12. The fusion protein of claim 11, wherein said second, heterologous protein is a therapeutic protein.

13. The fusion protein of claim 12, wherein said therapeutic protein is a toxin.

14. A targeted chimeric molecule comprising:
(a) at least a portion of a molecule, wherein said portion specifically binds to a target molecule, wherein said target molecule is selected from the group consisting of the proteins and the ribonucleotides encoded by the polynucleotides set forth in Table 1A and B, wherein said portion is sufficient to confer specific binding to the target molecule; and
(b) a therapeutic agent.

15. The targeted chimeric molecule of claim 14, wherein said therapeutic agent is a cytotoxic agent.

16. A method for delivering a therapeutic agent to a tissue of interest in a patient in need thereof, said method comprising the step of administering to said patient the targeted chimeric molecule of claim 14.

17. A method for eliminating a tissue in a patient in need thereof, said method comprising the step of administering to said patient a targeted chimeric molecule according to claim 15.

18. The method of claim 17, wherein said tissue is a cancerous tissue, and wherein said target molecule is selected from the group consisting of the ribonucleotides and the proteins encoded by the polynucleotides set forth in Table 1B.

19. A targeted therapeutic liposome comprising:
(a) at least a portion of molecule, wherein said portion specifically binds to a target molecule, wherein said target molecule is selected from the group consisting of the ribonucleotides and the proteins encoded by the polynucleotides set forth in Table 1A and B, wherein said portion is sufficient to confer specific binding to the target molecule; and
(b) a therapeutic liposome.

20. The targeted therapeutic liposome of claim 19, wherein said therapeutic liposome comprises a cytotoxic agent.

21. A method for delivering a therapeutic agent to a specific tissue in a patient in need thereof, said method comprising the step of administering to said patient a targeted therapeutic liposome according to claim 19.

22. A method for eliminating a tissue in a patient in need thereof, said method comprising the step of administering to said patient a targeted therapeutic liposome according to claim 20.

23. The method of claim 22, wherein said tissue is a cancerous tissue, and wherein said target molecule is selected from the group consisting of the ribonucleotides and the proteins encoded by the polynucleotides set forth in Table 1B.

24. A method for arresting cancer, said method comprising inhibiting a cancer-associated molecule according to Table 1B.

25. The method of claim 24, wherein said cancer-associated molecule is inhibited using an antisense polynucleotide.

26. The method of claim 24, wherein said cancer-associated molecule is inhibited using an antibody that specifically binds to said cancer-associated molecule.

27. A method for identifying a modulator of cancer development in a cell, said method comprising the steps of:
(a) culturing said cell in the presence of the modulator to form a first cell culture;
(b) contacting RNA or cDNA from said first cell culture with a probe which comprises a polynucleotide sequence selected from the sequences set forth in Table 1B;
(c) determining whether the amount of said probe which hybridizes to the RNA or cDNA from said first cell culture is increased or decreased relative to the amount of said probe which hybridizes to RNA or cDNA from a second cell culture grown in the absence of said modulator; and
(d) detecting the presence or absence of a decreased proliferative potential, transformation or malignancy in said first cell culture relative to said second cell culture.

28. A method for modulating cancer development in a patient in need thereof, said method comprising the step of administering to said patient a modulator of cancer development according to claim 27, wherein said modulator decreases the expression of a polynucleotide sequence selected from the group consisting of the sequences set forth in Table 1B.
